SEQUENCE LISTING (5 sequences)

SEQ ID NO: 1 - DNA, 7944 bp, Artificial Sequence
  Plasmid pDLZ6 encoding Homo sapiens BDD FVIII
tggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctctttctaagtaaacagtacatgaacctttaccccgttgctcggcaacggcctggtctgtgccaagtgtttgctgacgcaacccccactggctggggcttggccataggccatcagcgcatgcggatctcagtgtggttttgcaagaggaagcaaaaagcctctccacccaggcctggaatgtttccacccaatgtcgagcagtgtggttttgcaagaggaagcaaaaagcctctccacccaggcctggactcgagagcttcgaccaccatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgactcgagcgagttcttctgaggggatcggcaataaaaagacagaataaaacgcacgggtgttgggtcgtttgttcggatccagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggctgcaggggggggggggggggggt

SEQ ID NO: 2 - protein, 1471 aa, Homo sapiens B-domain deleted factor VIII
  Homo sapiens BDD FVIII
MQIELSTCFFLCLLRFCFSATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 3 - DNA, 7914 bp, Artificial Sequence
  rAAV vector with canine B-domain deleted factor VIII
tggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctctttctaagtaaacagtacatgaacctttaccccgttgctcggcaacggcctggtctgtgccaagtgtttgctgacgcaacccccactggctggggcttggccataggccatcagcgcatgcggatctcagtgtggttttgcaagaggaagcaaaaagcctctccacccaggcctggaatgtttccacccaatgtcgagcagtgtggttttgcaagaggaagcaaaaagcctctccacccaggcctggactcgacctcgagagtacttctagaaatacgagccatgcaagtagagctctacacctgctgctttctgtgccttttgcccttcagccttagtgccaccagaaaatactacctcggtgcagtggaactgtcctgggactatatgcaaagtgacctgctcagtgcgctgcacgcggatacaagcttttcttccagggtgccaggatctttgccactcaccacgtcagtcacgtacagaaagactgtgtttgtagagtttacagatgaccttttcaacattgccaagcccaggccaccgtggatgggcctgctgggtcctaccatccaggctgaggtttatgacacagtggtcattgtccttaagaacatggcttctcatcctgtcagccttcacgctgttggtgtatcctattggaaagcttctgaaggtgctgagtatgaggatcagaccagccaaaaggagaaggaagatgataatgtcattcctggtgaaagccatacctatgtctggcaggtcctgaaagagaatggcccaatggcctctgatccaccatgtctcacctactcatatttttcacacgtggacctggtgaaagacctgaattcaggcctcattggagccctgctggtttgcaaagaagggagtctggccaaagaaaggacacagaccttgcaggaatttgtcctactttttgctgtatttgatgaagggaaaagttggcactcagaaacaaatgcgtctttgacacaggctgaggcccagcatgagctgcacaccatcaatggctatgtaaacaggtctctgccaggtcttactgtgtgtcacaagagatcagtctattggcatgtgattggaatgggcaccacccccgaagtgcactcaatttttctcgaaggtcacacatttcttgtgaggaaccaccgccaggcctccttggagatctcaccaattactttccttactgctcagacattcctgatggaccttggccagtttctactgttttgtcatatcccttcccatcaacatgatggtatggaagcttatgtcaaagtagatagctgcccagaggaaccccagctgcgcatgaaaaataatgaagataaagattatgatgatggtctttatgattctgacatggacgtagttagctttgatgacgacagctcttctccctttatccaaatccgctcagttgccaagaagcatcctaaaacttgggtccactatattgctgctgaggaggaggactgggactatgctccctcaggccccacccccaatgatagaagtcataaaaatctgtatttgaacaatggtcctcagcggattggtaagaagtacaaaaaagtccgatttgtggcatacacagatgagacatttaagactcgtgaagctattcagtatgaatcaggaatcctgggacctttactttatggagaagttggagacacactgctgattatatttaagaatcaagccagccggccatataacatctaccctcatgggatcaattatgtcactcctctgcacacagggagattgccaaaaggtgtgaaacatttgaaagatatgccaattctgccgggagagatattcaagtataaatggacagtgaccgtagaagatggaccaactaaatcagatcctcggtgcctgacccgatattactcaagcttcattaatctggagagagatctagcttcaggactcattggccctcttctcatctgctacaaagaatctgtagatcaaagaggaaaccagatgatgtcagacaagagaaatgtcatcctgttttctgtatttgatgagaatcgaagctggtacctcacagagaatatgcagcgcttcctccccaatgcagatgtagtgcagccccatgacccagagttccaactctctaacatcatgcacagcatcaatggctatgtttttgacaacttgcagctgtcagtttgtttgcatgaggtggcgtactggtacattctaagtgttggagcacaaactgacttcctgtctgtcttcttctctggatataccttcaaacacaaaatggtctatgaagacacacttaccctcttcccattctcaggagaaactgtcttcatgtcaatggaaaacccaggtctgtgggttctggggtgccacaactcagactttcggaacagaggcatgacagccttactgaaggtttctagttgtaacaggaacattgatgattattatgaggacacatacgaagatattccaactcccctgctaaatgaaaacaatgtaattaaacctagaagcttctcccagaattcaaggcaccctagcactaaggaaaagcaattgaaaatgaagagagaagattttgacatctacggcgactatgaaaatcagggcctccgcagctttcaaaagaaaacacgacactatttcattgctgcagtggagcgtctctgggattatgggatgagtagatctccccatatactaagaaacagggctcaaagtggggatgtccagcagttcaagaaggtggttttccaggaatttactgatggatcctttactcagcccttataccgtggagaactgaatgaacacttgggactcttggggccatatataagagcagaagttgaagacaatatcgtggtaactttcaaaaaccaggcctctcgtccctactccttctattctagtcttatttcttatgacgaagatgagggacaaggagcagaacctagaagaaagtttgtcaaccctaatgaaaccaaaatttacttttggaaagtgcagcatcatatggcacccactaaagatgagtttgactgcaaagcctgggcttatttttctgatgttgatttggagaaagatgtgcactcaggcttgattggaccccttctgatctgccgcagtaacacactgaaccctgctcatgggagacaagtgacagtgcaggagtttgccctggttttcactatattcgatgagactaagagctggtacttcactgaaaacctggaaaggaactgtagagctccctgcaatgtccagaaggaggaccctactctaaaagaaaacttccgcttccatgcaatcaacggctatgtgaaggatacactccctggcttagtaatggctcaggatcaaaaggttcgatggtatctgctcagcatgggcagcaacgaaaacattcattccattcacttcagtggacatgtgttcactgtacggaaaaaagaggaatataaaatggcagtctacaacctctatccaggtgtttttgagactgtggaaatgctaccatcccaagttggaatctggcggatagaatgccttatcggcgagcacctgcaagccgggatgagcactctgtttctggtgtacagcaagaagtgtcagactccactggggatggcttccggacacattagagattttcagattacagcttcaggacaatatggacagtgggccccaaagctggccagacttcattattccggatcaatcaatgcctggagcaccaaggatcccttttcctggatcaaggtggatctcttggcaccgatgattattcacggcatcatgacccagggggcccgccagaagttctccagcctctacgtgtctcagtttatcatcatgtacagtctggatggcaacaagtggcacagttaccgagggaattccacggggaccttaatggtcttctttggcaacgtggattcatctgggatcaaacacaatatttttaaccctccgattattgctcagtacatccgtttgcacccaacccattacagcatccgcagcactcttcgcatggagctcttgggctgtgacttcaacagttgcagcatgccgctggggatggagagtaaagcaatatcagatgctcagatcactgcctcgtcctacctaagcagtatgcttgccacttggtctccttcccaagcccggctgcacctgcagggcaggactaatgcctggagacctcaggcaaataacccaaaagagtggctgcaagtggacttccggaagaccatgaaagtcacaggaataaccacccagggggtgaaatctctcctcatcagcatgtatgtgaaggagttcctcatctccagtagtcaagatggccataactggactctgtttcttcagaatggcaaagtcaaggtcttccagggaaaccgggactcctccacgcctgtgcggaaccgtctcgaacccccgctggtggctcgctacgtgcgcctgcacccgcagagctgggcgcaccacatcgccctgaggctggaggtcctgggctgcgacacccagcagcccgcctgacccgcgcctctgcggccctgtctcccctgcctccctgccctgtccccgcggcttcccatcaagcttatcgataccgtcgagcgagttcttctgaggggatcggcaataaaaagacagaataaaacgcacgggtgttgggtcgtttgttcggatccagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggctgcaggggggggggggggggggt

SEQ ID NO: 4 - protein, 1431 aa, canine B-domain deleted factor VIII
MQVELYTCCFLCLLPFSLSATRKYYLGAVELSWDYMQSDLLSALHADTSFSSRVPGSLPLTTSVTYRKTVFVEFTDDLFNIAKPRPPWMGLLGPTIQAEVYDTVVIVLKNMASHPVSLHAVGVSYWKASEGAEYEDQTSQKEKEDDNVIPGESHTYVWQVLKENGPMASDPPCLTYSYFSHVDLVKDLNSGLIGALLVCKEGSLAKERTQTLQEFVLLFAVFDEGKSWHSETNASLTQAEAQHELHTINGYVNRSLPGLTVCHKRSVYWHVIGMGTTPEVHSIFLEGHTFLVRNHRQASLEISPITFLTAQTFLMDLGQFLLFCHIPSHQHDGMEAYVKVDSCPEEPQLRMKNNEDKDYDDGLYDSDMDVVSFDDDSSSPFIQIRSVAKKHPKTWVHYIAAEEEDWDYAPSGPTPNDRSHKNLYLNNGPQRIGKKYKKVRFVAYTDETFKTREAIQYESGILGPLLYGEVGDTLLIIFKNQASRPYNIYPHGINYVTPLHTGRLPKGVKHLKDMPILPGEIFKYKWTVTVEDGPTKSDPRCLTRYYSSFINLERDLASGLIGPLLICYKESVDQRGNQMMSDKRNVILFSVFDENRSWYLTENMQRFLPNADVVQPHDPEFQLSNIMHSINGYVFDNLQLSVCLHEVAYWYILSVGAQTDFLSVFFSGYTFKHKMVYEDTLTLFPFSGETVFMSMENPGLWVLGCHNSDFRNRGMTALLKVSSCNRNIDDYYEDTYEDIPTPLLNENNVIKPRSFSQNSRHPSTKEKQLKMKREDFDIYGDYENQGLRSFQKKTRHYFIAAVERLWDYGMSRSPHILRNRAQSGDVQQFKKVVFQEFTDGSFTQPLYRGELNEHLGLLGPYIRAEVEDNIVVTFKNQASRPYSFYSSLISYDEDEGQGAEPRRKFVNPNETKIYFWKVQHHMAPTKDEFDCKAWAYFSDVDLEKDVHSGLIGPLLICRSNTLNPAHGRQVTVQEFALVFTIFDETKSWYFTENLERNCRAPCNVQKEDPTLKENFRFHAINGYVKDTLPGLVMAQDQKVRWYLLSMGSNENIHSIHFSGHVFTVRKKEEYKMAVYNLYPGVFETVEMLPSQVGIWRIECLIGEHLQAGMSTLFLVYSKKCQTPLGMASGHIRDFQITASGQYGQWAPKLARLHYSGSINAWSTKDPFSWIKVDLLAPMIIHGIMTQGARQKFSSLYVSQFIIMYSLDGNKWHSYRGNSTGTLMVFFGNVDSSGIKHNIFNPPIIAQYIRLHPTHYSIRSTLRMELLGCDFNSCSMPLGMESKAISDAQITASSYLSSMLATWSPSQARLHLQGRTNAWRPQANNPKEWLQVDFRKTMKVTGITTQGVKSLLISMYVKEFLISSSQDGHNWTLFLQNGKVKVFQGNRDSSTPVRNRLEPPLVARYVRLHPQSWAHHIALRLEVLGCDTQQPA

SEQ ID NO: 5 - DNA, 12 bp, Artificial Sequence
  liver-preferred CAAT box binding sites for C/EBP proteins
gattgcgcaatc

(19) United States
(12) Patent Application Publication
(10) Pub. No.: US 2002/0131956 A1
(43) Pub. Date: Sep. 19, 2002

(54) Adeno-associated virus vectors encoding factor VIII and methods of using the same

(75) Inventors: Christopher E. Walsh, Chapel Hill, NC (US); Hengjun Chao, Carrboro, NC (US); Haim Burstein, Redmond, WA (US); Carmel Lynch, Kenmore, WA (US); Tony Stepan, Seattle, WA (US); Keith Munson, Seattle, WA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: The University of North Carolina, Chapel Hill, NC (US)

(21) Appl. No.: 10/095,718

(22) Filed: Mar. 12, 2002

Related U.S. Application Data
(63) Continuation of application No. 09/689,430, filed on Oct. 12, 2000.
(60) Provisional application No. 60/158,780, filed on Oct. 12, 1999.

(51) Int. Cl. A61K 48/00; C12N 7/00; C12N 15/861

(52) U.S. Cl. 424/93.2; 435/235.1; 435/456

(57) ABSTRACT

The present invention provides recombinant adeno-associated virus (rAAV) vectors comprising a heterologous nucleotide sequence encoding factor VIII (factor VIII). In preferred embodiments, the factor VIII is a B-domain deleted factor VIII. Also provided are methods of producing a high titer stock of the inventive rAAV/factor VIII vectors. Another aspect of the invention is a method of delivering a nucleotide sequence encoding factor VIII to a cell, preferably for subsequent administration to a subject. The present invention further provides methods of administering rAAV/factor VIII to a subject, e.g., for the treatment of hemophilia. The rAAV vector may be administered by any route, but is preferably administered to the liver.

### CROSS-REFERENCE TO RELATED APPLICATIONS

The present application claims the benefit of U.S. Provisional Application Serial No. 60/158,780 filed Oct. 12, 1999, entitled “Adeno-Associated Virus Vectors Encoding Factor VIII and Methods of Using the Same,” the contents of which are herein incorporated by reference in their entirety.

### FIELD OF THE INVENTION

This invention relates to reagents and methods for providing Factor VIII, and more particularly relates to viral reagents and methods for providing Factor VIII.

### BACKGROUND OF THE INVENTION

Hemophilia A is an inherited sex-linked bleeding disease resulting from deficiency of coagulation factor VIII (factor VIII). Hemophilia A comprises the majority of hemophilia patients (80%) with an incidence of 1 in 5-10,000 live males births (Antonarakis et al. (1998) Haemophilia 4:1). Hemophilia patients suffer from spontaneous bleeding into the large joints, soft tissue, and are at risk for intracranial hemorrhage. Recurrent episodes of joint bleeding are the most frequent manifestation of the disease leading to crippling arthropathy, particularly in severely affected patients.

Gene therapy is an attractive alternative for the treatment of hemophilia A patients. Persistent expression of human factor VIII would make a profound impact on treatment of hemophilia A patients even at levels less than therapeutic levels (approximately equal to or greater than 5% of normal). Both retroviral and adenoviral vectors have been used to deliver factor VIII cDNA (Dwarki et al. (1995) Proc. Nat. Acad. Sci. USA 92:1023; Connelly et al. (1998) Blood 91:3273; Connelly et al. (1996) Blood 87:4671). Moloney murine leukemia virus (MoMLv) amphotropic vectors suffer from poor transduction of post-mitotic cells (Dwarki et al. (1995) Proc. Nat. Acad. Sci. USA 92:1023). Adenovirus carrying the human factor VIII cDNA directed to the liver express high-level factor VIII in animal models. However expression wanes with time due to the well-characterized cell-mediated immune response to the vector (Connelly et al. (1996) Blood 87:4671; Connelly et al. (1996) Blood 88:3846). Such immune responses can have serious consequences to the recipient. Immune responses result in inflammation, cell death, and even death of the patient.

Adeno-associated virus is a nonpathogenic defective parvovirus capable of infecting a broad range of mitotic or post-mitotic cells (Rabinowitz et al. (1998) Current Opinion in Biotechnology 9:470). rAAV has been shown to be capable of expressing a functional FIX gene persistently in a large animal model (Snyder et al. (1999) Nature Medicine 5:64), where factor VIII and FIX are synthesized (Wion et al. (1985) Nature 317:726; Zelechowska et al. (1 985) Nature 317:729).

A disadvantage of rAAV vectors is their restricted packaging capacity (Dong et al. (1996) Human Gene Therapy, 7:2101). Wild-type (wt) AAV is a 4.6 kb linear single-stranded DNA virus. The total size of the AAV vector influences the efficiency of its packaging into AAV virions. Dong et al. determined the packaging efficiencies of AAV vectors by quantitating the DNA content of viral particles and assaying the efficiency of AAV virions to transfer the CAT gene into HeLa cells. Efficient packaging as determined by Dong et al. includes particles that contain and express the transgene. The results demonstrate that the packaging efficiency of AAV is affected by the length of the genome.

The human factor VIII gene comprises a central B domain core flanked by the amino A1 and A2 domains and carboxyl A3, C1, and C2 domains. The B domain can be deleted without any significant effect on specific procoagulant activity (Pittman et al. (1993) Blood 81:2925). However, even B-domain deleted human factor VIII cDNA (B-domain deleted human factor VIII) is not thought feasible for testing in rAAV (Pittman et al. (1993) Blood 81:2925), as its 4.4 kb size is believed to preclude its efficient packaging within the limited confines of a rAAV vector (Kay and High (1999) Proc. Natl. Acad. Sci. USA 96:9973). Thus, it is felt that production of high-titer AAV B-domain deleted human factor VIII vector would be very difficult (Kay and Russell (1999) Blood 94:864).

Somatic cell gene therapy to treat hemophilia A is further complicated by difficulties attendant to expression of the factor VIII gene. Persistent human factor VIII expression has been demonstrated to be hampered by poor transcription efficiency of the human factor VIII gene (Connelly et al. (1996) Blood 91:3846; Rabinowitz et al. (1998) Current Opinion in Biotechnology 9:470), inefficient secretion of factor VIII protein (Snyder et al. (1999) Nature Medicine 5:64; Wion et al. (1985) Nature 31 7:726), and the relatively short half-life of the factor VIII protein (t_{½}˜12 hours; Wion et al. (1985) Nature 317:726; Zelechowska et al. (1985) Nature 317:729).

Accordingly, there remains a need in the art for improved reagents and methods for treating hemophilia A.

### SUMMARY OF THE INVENTION

Compositions and methods for the expression of a biologically active factor VIII (factor VIII) protein in a subject are provided. The compositions and methods are useful in the treatment of coagulation disorders, particularly hemophilia A, in a subject. The compositions include a recombinant AAV (rAAV) vector comprising a nucleotide sequence encoding B-domain deleted factor VIII operably linked with at least one enhancer and at least one promoter. In some embodiments, the AAV ITR is operably linked to the nucleotide sequence encoding the B-domain deleted factor VIII, such that the ITR drives the expression of the B-domain deleted factor VIII transgene. The vector may also comprise a transcription factor binding site and/or a termination region. Optionally, spacer DNA can be included within the cassette. The rAAV vector of the invention encodes a biologically-active B-domain deleted factor VIII protein that may be administered in vivo to achieve long-term expression of therapeutic levels of factor VIII protein. Accordingly, the present invention utilizes the many advantages of rAAV vectors, while overcoming the constraints imposed by the limited packaging capacity of the AAV capsid.

Another aspect of the invention is an rAAV vector comprising a heterologous nucleotide sequence encoding a B-domain deleted factor VIII selected from the group consisting of: (a) about nucleotides 419 to 4835 of FIG. 1 (also shown in SEQ ID NO:1), (b) a nucleotide sequence that hybridizes to the nucleotide sequence of (a) under conditions of high stringency and which encodes a B-domain deleted factor VIII, and (c) a nucleotide sequence that that differs from the nucleotide sequences of (a) and (b) above due to the degeneracy of the genetic code, and which encodes a B-domain deleted factor VIII.

The invention also provides methods of delivering a heterologous nucleotide sequence encoding B-domain deleted factor VIII to cells in vitro and in vivo. Accordingly in one embodiment, a method is provided for delivering a nucleotide sequence encoding B-doamin deleted factor VIII to a cell, the method comprising contacting the cell with a rAAV vector comprising a heterologous nucleotide sequence encoding factor VIII operably linked with a liver-preferred expression control element. The contacting may be carried out in vitro or in vivo.

A further embodiment is a method of delivering a nucleotide sequence encoding a B-domain deleted factor VIII to a cell comprising contacting the cell with the rAAV vector of the invention. The rAAV vector comprising a heterologous nucleotide sequence encoding a B-domain deleted factor VIII selected from the group consisting of: (a) about nucleotides 419 to 4835 of FIG. 1 (also shown in SEQ ID NO:1), (b) a nucleotide sequence that hybridizes to the nucleotide sequence of (a) under conditions of high stringency and which encodes a B-domain deleted factor VIII, and (c) a nucleotide sequence that differs from the nucleotide sequences of (a) and (b) above due to the degeneracy of the genetic code, and which encodes a B-domain deleted factor VIII.

In yet a further aspect, the present invention provides a method of treating hemophilia A comprising administering to a hemophiliac subject a biologically effective amount of a rAAV vector comprising a heterologous nucleotide sequence encoding B-domain deleted factor VIII. Preferably, the encoded B-domain deleted factor VIII is expressed in a therapeutically effective amount.

In a further embodiment, the invention provides a method of treating hemophilia comprising administering a biologically effective amount of a rAAV comprising a heterologous nucleotide sequence encoding B-domain deleted factor VIII to a liver cell of a hemophiliac subject. Preferably, the encoded B-domain deleted factor VIII is expressed by the transduced liver cell and is secreted into the blood in a therapeutically effective amount.

As a still further embodiment, the present invention provides a method of administering factor VIII to a subject comprising administering a cell expressing factor VIII to the subject, wherein the cell has been produced by a method comprising contacting the cell with a recombinant adeno-associated virus (AAV) vector of the invention.

The present invention further provides a method of producing a high-titer stock of a rAAV vector comprising: (a) infecting a packaging cell with a rAAV vector comprising a heterologous nucleotide sequence encoding factor VIII, (b) allowing the rAAV genome to replicate and be encapsidated by the packaging cell, and (c) collecting the rAAV particles to form a rAAV stock. As indicated, the heterologous nucleotide sequence encoding B domain deleted factor VIII is operably linked with a liver-preferred expression control element. Also provided are high-titer virus stocks produced by the foregoing method.

Methods for the production of a stable cell line by infection with the rAAV vector of the invention are also provided. Such cell lines are generated by transfection with vector, selection, followed by cloning of individual colonies. Clones exhibiting high level replication of vector are then tested for production of infectious vector. The cell line is capable of expressing B domain deleted VIII.

Another aspect of the invention is a nucleotide sequence encoding factor VIII operably linked with a hepatitis virus expression control element. In some embodiments, this expression control element is from hepatitis B and comprises at least one of the enhancers selected from the hepatitis EnhI enhancer and the EnhII enhancer. The nucleotide sequence may further comprise at least one promoter and a polyadenylation sequence. In some embodiments, at least one promter is an AAV ITR. The invention also encompasses vectors comprising the nucleotide sequence encoding factor VIII operably linked with a hepatitis virus expression control element, and host cells containing this vector.

These and other aspects of the present invention are provided in more detail in the description of the invention below.

### BRIEF DESCRIPTION OF THE DRAWINGS

FIG. 1 provides the sequence of plasmid pDLZ6 encoding a human B-domain deleted factor VIII. This sequence is also set forth in SEQ ID NO:1. The expression cassette includes the left and right AAV inverted terminal repeats (ITR; about nucleotides 1-146 and 4916-5084), the hepatitis B virus EnhI enhancer (about nucleotides 150-278), spacer sequence (nucleotides 279-399), human B-domain deleted factor VIII (about nucleotides 419-4835), and the TK poly(A) sequence (about nucleotides 4840-4914). The amino acid sequence for human B-domain deleted factor VIII encoded by nucleotides 419-4835 (SEQ ID NO:2) is also shown.

FIG. 2 is a schematic representation of the rAAV/B-domain deleted human factor VIII constructs. The maps for the two rAAV constructs expressing B-domain deleted human factor VIII are shown: pDLZ2 (4965 bp including 2 ITRs, 107% of wt-AAV) and pDLZ6 (5089 bp including 2 ITRs, 109% of wt-AAV). ITR, AAV inverted terminal repeat; EnhI, Enhancer I of the HBV; NCS, spacer sequence; P(A), TK polyadenylation sequence.

FIG. 3 shows the replication and packaging of rAAV/B-domain deleted human factor VIII. Low molecular weight DNA (Hirt DNA) was isolated from rAAV/DLZ2, DLZ6, and DLZ8 (control) transduced HeLa and HepG2 cells, separated by agarose gel, and probed with B-domain deleted human factor VIII cDNA. From right to left: Control Lane, 1-HepG2+rAAV/DLZ8; 2-HeLa+rAAV/DLZ8; DLZ2: 1-HeLa+rAAV/DLZ2; 2-HepG2+rAAV/DLZ2; DLZ6: 1-HeLa+rAAV/DLZ6; 2-HepG2+rAAV/DLZ6; and uncoated rAAV/DLZ6 virion DNA.

FIG. 4 is a graphical representation of in vivo expression of rAAV/B-domain deleted human factor VIII in mice. Purified rAAV/DLZ6 virus was administered to the mice via the portal vein. ELISA was employed to determine human factor VIII level in the plasma and BIA was utilized to measure anti-human factor VIII inhibitor titer. Panel A. B-domain deleted human factor VIII antigen level and anti-human factor VIII inhibitor titer in the plasma of the mice (n=4) receiving 2×10¹¹ rAAV/DLZ6. Panel B. B-domain deleted human factor VIII antigen measurement of NOD/scid mice (n=4) receiving 1.5×10¹¹ rAAV/DLZ6. Solid line: human factor VIII antigen level, Dashed line: anti-B-domain deleted human factor VIII inhibitor titer.

FIG. 5 presents molecular analysis of the mice receiving injection of rAAV/DLZ6. Panel A. Diagram of the primers designed for the PCR. Panel B. DNA PCR-rAAV vectors distribution in mice via portal vein injection. A rAAV/DLZ6 unique 450 bp fragment was amplified by DNA PCR to test distribution of rAAV after hepatic injection. Negative control, Liver DNA of the control mouse. DNA samples of brain, spinal cord, muscle, bone marrow, heart, lungs, testis, lymph nodes, kidney, intestine, spleen from the mouse receiving high dose rAAV/DLZ6. Liver/LD: liver DNA from mouse receiving low dose rAAV/DLZ6. Liver HD: liver DNA from mouse receiving high dose rAAV/DLZ6. Standard curve-genomic DNA from control mouse liver with 5, 1, 0.2, 0.1, 0.01 and 0 genome copy equivalents of plasmid pDLZ6 per cell, respectively. Panel C. Diagram of the primers designed for RT/PCR. Panel D. RT-PCR analysis of total RNA isolated from control and experimental animals. Primers were designed to amplify a 534 bp B-domain deleted-human factor VIII specific fragment. RT control employed RNA isolated from the mouse liver receiving high dose rAAV/DLZ6. The negative control used RNA isolated from control animal. RNA samples of muscle, brain, lymph nodes, testis, kidney and spleen were from the mouse receiving high dose rAAV/DLZ6. LD: liver RNA isolated from mouse receiving low dose AAV/DLZ6. HD: liver RNA isolated from mouse receiving high dose rAAV/DLZ6. Panel E. Diagram of the restriction digestion using Sph I. Panel F. Southern blot analysis of high molecular weight genomic DNA and Hirt DNA isolated from experimental animals. Standard curve: genomic DNA from control mouse liver with 5, 1, 0.2, and 0.02 genome copy equivalents of plasmid pDLZ6 per cell, respectively. HMW genomic DNA and low molecular wt liver DNA (HIRT) isolated from animals receiving high dose rAAV/DLZ6.

FIG. 6 provides the sequence of plasmid pDLZ10 (SEQ ID NO:3) encoding a canine B-domain deleted factor VIII. The expression cassette includes the left and right AAV inverted terminal repeats (ITR; nucleotides 1-144 and 4885-5048), the hepatitis B virus EnhI enhancer (nucleotides 149-278), spacer sequence (nucleotides 279-399), canine B-domain deleted factor VIII (about nucleotides 428-4790), and the TK poly(A) sequence (nucleotides 4804-4884). The amino acid sequence for canine B-domain deleted factor VIII encoded by nucleotides 428-4790 is also shown in this figure and in SEQ ID NO:4.

### DETAILED DESCRIPTION OF THE INVENTION

The invention provides compositions and methods to alleviate the symptoms associated with factor VIII deficiency. Compositions include rAAV vectors comprising a nucleotide sequence encoding a B-domain deleted factor VIII protein operably linked with at least one enhancer and at least one promoter. In some embodiments, the vector comprises a liver-preferred expression control element. Spacer DNA and a 3′ termination region may be optionally included within the cassette.

While the invention is not bound by any mechanism of action, it is believed that in the preferred embodiments, the ITR region or regions of the AAV serves as a promoter to drive expression of the factor VIII nucleotide sequence. That is, at least one of the inverted terminal repeats (ITRs) found at each end of the AAV genome is used to drive expression of the B-domain deleted factor VIII sequence. See, for example, U.S. Pat. No. 5,866,696, herein incorporated in its entirety by reference.

The following definitions are provided to be used to understand the invention as set forth herein and in the attached claims.

An “expression control element” is a polynucleotide sequence, preferably a DNA sequence, which increases transcription of an operably linked or operably linked polynucleotide in a host cell that allows that expression control element to function. An expression control element can comprise an enhancer, promoter, and/or a transcription factor binding site. A liver-preferred transcriptional regulatory element is an expression control element that increases transcription of an operably linked polynucleotide sequence in a liver cell in comparison with a non-liver cell.

“Factor VIII-associated disorders” are those disorders or diseases that are associated with, result from, and/or occur in response to, insufficient levels of factor VIII. Such disorders include, but are not limited to, hemophilia A.

The terms “polypeptide” “peptide” and “protein” are used interchangeably herein to refer to polymers of amino acids of any length. The terms also encompass an amino acid polymer that has been modified; for example, disulfide bond formation, glycosylation, lipidation, or conjugation with a labeling component.

The terms “polynucleotide”, “nucleotide sequence”, and “nucleic acid”, used interchangeably herein, refer to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

“AAV” is an abbreviation for adeno-associated virus, and may be used to refer to the virus itself or derivatives thereof. The term covers all subtypes and both naturally occurring and recombinant forms, except where required otherwise. “AAV” refers to adeno-associated virus in both the wild-type and the recombinant form (rAAV) and encompasses mutant forms of AAV. The term AAV further includes, but is not limited to, AAV type 1, AAV type 2, AAV type 3, AAV type 4, AAV type 5, AAV type 6, AAV type 7, avian AAV, bovine AAV, canine AAV, equine AAV, and ovine AAV (see, e.g., Fields et al., Volume 2, Chapter 69 (3d ed., Lippincott-Raven Publishers). In a preferred embodiment, the AAV used in the present invention is AAV type 2.

By “adeno-associated virus inverted terminal repeats” or “AAV ITRs” is meant the palindromic regions found at each end of the AAV genome. The ITRs function together in cis as origins of DNA replication and as packaging signals for the virus. For use with the present invention, flanking AAV ITRs are positioned 5′ and 3′ of a cassette comprising a B domain deleted factor VIII coding sequence operably linked with an enhancer and optionally spacer DNA or promoter elements. In some embodiments, the AAV ITR is operably linked to the B-domain deleted factor VIII encoding nucleotide sequence such that it drives expression of this sequence.

The nucleotide sequences of AAV ITR regions are known. See, e.g., Kotin, R. M. (1994) Human Gene Therapy 5:793-801; Bems, “Parvoviridae and Their Replication,” in Fundamental Virology, 2d ed. (ed. Fields and Knipe) for the AAV-2 sequence. As used herein, an “AAV ITR” need not have the wild-type nucleotide sequence depicted, but may be altered, e.g., by the insertion, deletion or substitution of nucleotides. Additionally, the AAV ITR may be derived from any of several AAV serotypes, including without limitation, AAV-1, AAV-2, AAV-3, AAV-4, AAV-5, AAV-6, AAV-7, etc. The 5′ and 3′ ITRs flanking a selected heterologous nucleotide sequence comprising a factor VIII coding sequence need not necessarily be identical or derived from the same AAV serotype or isolate, so long as they function as intended, i.e., to allow for the integration of the associated heterologous sequence into the target cell genome when the rep gene is present (either on the same or on a different vector), or when the Rep expression product is present in the target cell. Recent evidence suggests that a single ITR can be sufficient to carry out the functions normally associated with configurations comprising two ITRs (U.S. Pat. No. 5,478,745), and vector constructs with only one ITR can thus be employed in conjunction with the packaging and production methods described herein.

A “biologically effective” amount of an rAAV vector of the invention is an amount that is sufficient to result in transduction and expression of the heterologous nucleotide sequence encoding the B-domain deleted factor VIII by at least one cell in the target tissue or organ.

An “rAAV vector”, “rAAV virus”, or “rAAV viral particle” as used herein contains at least one AAV capsid protein (preferably by all of the capsid proteins of a wild-type AAV) and an encapsidated rAAV comprising a polynucleotide sequence not of AAV origin (i.e., a polynucleotide heterologous to AAV), typically a sequence of interest for the genetic transformation of a cell. The heterologous polynucleotide is flanked by at least one, preferably two, AAV inverted terminal repeat sequences (ITRs).

“Packaging” refers to a series of intracellular events that result in the assembly and encapsidation of an AAV particle or rAAV particle. In the case of the rAAV particle, packaging refers to the assembly and encapsidation of the rAAV particle including the transgene.

AAV “rep” and “cap” genes refer to polynucleotide sequences encoding replication and encapsidation proteins of adeno-associated virus. They have been found in all AAV serotypes examined, and are described below and in the art. AAV rep and cap are referred to herein as AAV “packaging genes”.

A “helper virus” for AAV refers to a virus that allows AAV to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

An “infectious” virus or viral particle is one that comprises a polynucleotide component which it is capable of delivering into a cell for which the viral species is trophic. The term does not necessarily imply any replication capacity of the virus. Assays for counting infectious viral particles are described in the art.

A “replication-competent” virus (e.g., a replication-competent AAV, sometimes abbreviated as “RCA”) refers to a phenotypically wild-type virus that is infectious, and is also capable of being replicated in an infected cell (i.e., in the presence of a helper virus or helper virus functions). In the case of AAV, replication competence generally requires the presence of functional AAV packaging genes. Preferred rAAV vectors as described herein are replication-incompetent in mammalian cells (especially in human cells) by virtue of the lack of one or more AAV packaging genes. Preferably, such rAAV vectors lack any AAV packaging gene sequences in order to minimize the possibility that RCA are generated by recombination between AAV packaging genes and an rAAV vector.

A “gene” refers to a polynucleotide containing at least one open reading frame that is capable of encoding a particular protein after being transcribed and translated.

“Expression”, as used herein, refers to the transcription and/or translation of a gene.

“Recombinant”, as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

“Operatively linked” or “operably linked” or “operably associated” refers to a juxtaposition of genetic elements, wherein the elements are in a relationship permitting them to operate in the expected manner. For instance, a promoter is operably linked to a coding region if the promoter helps initiate transcription of the coding sequence. There may be intervening residues between the promoter and coding region so long as this functional relationship is maintained.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operably linked to a coding sequence with which it is not naturally found linked is a heterologous promoter.

“Genetic alteration” refers to a process wherein a genetic element is introduced into a cell other than by mitosis or meiosis. The element may be heterologous to the cell, or it may be an additional copy or improved version of an element already present in the cell. Genetic alteration may be effected, for example, by transfecting a cell with a recombinant plasmid or other polynucleotide through any process known in the art, such as electroporation, calcium phosphate precipitation, or contacting with a polynucleotide-liposome complex. Genetic alteration may also be effected, for example, by transduction or infection with a DNA or RNA virus or viral vector. Preferably, the genetic element is introduced into a chromosome or mini-chromosome in the cell; but any alteration that changes the phenotype and/or genotype of the cell and its progeny is included in this term.

A cell is said to be “stably” altered, transduced, or transformed with a genetic sequence if the sequence is available to perform its function during extended culture of the cell in vitro. In preferred examples, such a cell is “inheritably” altered in that a genetic alteration is introduced which is also inheritable by progeny of the altered cell.

“Stable integration” of a polynucleotide into a cell means that the polynucleotide has been integrated into a replicon that tends to be stably maintained in the cell. Although episomes such as plasmids can sometimes be maintained for many generations, genetic material carried episomally is generally more susceptible to loss than chromosomally-integrated material. However, maintenance of a polynucleotide can often be effected by incorporating a selectable marker into or adjacent to a polynucleotide, and then maintaining cells carrying the polynucleotide under selective pressure. In some cases, sequences cannot be effectively maintained stably unless they have become integrated into a chromosome; and, therefore, selection for retention of a sequence comprising a selectable marker can result in the selection of cells in which the marker has become stably-integrated into a chromosome. Antibiotic resistance genes can be conveniently employed as such selectable markers, as is well known in the art. Typically, stably-integrated polynucleotides would be expected to be maintained on average for at least about twenty generations, preferably at least about one hundred generations, still more preferably they would be maintained permanently. The chromatin structure of eukaryotic chromosomes can also influence the level of expression of an integrated polynucleotide. Having the genes carried on stably-maintained episomes can be particularly useful where it is desired to have multiple stably-maintained copies of a particular gene. The selection of stable cell lines having properties that are particularly desirable in the context of the present invention are described and illustrated below.

An “isolated” plasmid, virus, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this invention are increasingly more preferred. Thus, for example, a 2-fold enrichment is preferred, 10-fold enrichment is more preferred, 100-fold enrichment is more preferred, 1000-fold enrichment is even more preferred.

A preparation of rAAV is said to be “substantially free” of helper virus if the ratio of infectious rAAV particles to infectious helper virus particles is at least about 10²:1; preferably at least about 10⁴:1, more preferably at least about 10⁶:1; still more preferably at least about 10⁸:1. Preparations are also preferably free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g. the appearance of bands other than those corresponding to the AAV capsid proteins VP1, VP2 and VP3).

A “host cell” includes an individual cell or cell culture which can be or has been a recipient for vector(s) or for incorporation of polynucleotides and/or proteins. Host cells include progeny of a single host cell, and the progeny may not necessarily be completely identical (in morphology or in genomic of total DNA complement) to the original parent cell due to natural, accidental, or deliberate mutation. A host cell includes cells transfected in vivo with a polynucleotide(s) of this invention.

By “liver cell” is intended any cell type found in liver organs, including, but not limited to parenchyma cells, nonparenchyma cells, endothelial cells, epithelial cells, etc.

“Transformation” or “transfection” refers to the insertion of an exogenous polynucleotide into a host cell, irrespective of the method used for the insertion, for example, lipofection, transduction, infection or electroporation. The exogenous polynucleotide may be maintained as a non-integrated vector, for example, a plasmid, or alternatively, may be integrated into the host cell genome.

An “individual” or “subject” refers to vertebrates, particularly members of a mammalian species, and includes, but is not limited to, domestic animals, sports animals, rodents and primates, including humans.

As used herein, “in conjunction with” refers to administration of one treatment modality in addition to another treatment modality, such as administration of an rAAV as described herein to a subject in addition to the delivery of factor VIII (in polypeptide form) to the same subject. As such, “in conjunction with” refers to administration of one treatment modality before, during or after delivery of the other treatment modality to the subject.

As used herein, “treatment” is an approach for obtaining beneficial or desired clinical results. For purposes of this invention, beneficial or desired clinical results include, but are not limited to, alleviation of at least one symptom, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples.

“Palliating” a disease means that the extent and/or undesirable clinical manifestations of a disease state are lessened and/or time course of the progression is slowed or lengthened, as compared to not administering rAAV vectors of the present invention.

As indicated, spacer DNA may be included within the construct of the invention. By “spacer DNA” is intended nonsense DNA that does not encode a protein and does not act as a promoter or promoter element. That is, spacer DNA may be utilized to provide any spatial requirements for the expression of the factor VIII nucleic acid molecule. The size or length of the spacer DNA may vary from a few nucleotides to several hundred nucleotides. The length of the spacer DNA will be limited by the size of the nucleotide sequence of the factor VIII to be expressed and the enhancer element, recognizing the size limitations of the rAAV vector.

By “titer” is intended the number of infectious viral units per volume of fluid.

By “high titer rAAV stock” is intended a stock of viral particles as produced from a production system, without artificial manipulation. “Without artificial manipulation” means that the number of viral particles has not been manipulated by pooling, multiple runs, or other concentration means. For purposes of the invention, one plate of cells, having about 2×10⁷ cells, will generate approximately 2 to 3×10¹¹ particles. These numbers can be scaled up appropriately. Of the number of viral particles produced, 1% will be functional virus. That is, 1 in 100 will express the factor VIII protein. Thus, approximately 2×10⁹ infectious virus particles in the preparation are functional. About 90-100%, of these express the transgene.

By “infectious units” is intended the smallest unit that causes a detectable effect when placed with a susceptible host. Assays for the determination of infectious units are known. For example, in one method used in the invention, virus is replicated on reporter cells in the presence of adenovirus and wild type AAV. After replication, DNA is obtained from the cells, probed for factor VIII coding sequence. In this manner, the number of rAAV in the cells can be determined.

To measure the total number of particles, cells can be probed with a viral nucleotide sequence. In the methods of the invention, the rAAV/factor VIII vector comprises about 90 to 99.9%, preferably about 99 to about 99.99% of the total particles. Wild type virus accounts for less than 0.01% of the total particles. Of these 99.9% of the particles obtained, 1 in 100, or 1% will be functional virus, that is will be virus that expresses the B-domain deleted factor VIII transgene.

The present invention is based, in part, on the unexpected finding that a biologically active B-domain deleted factor VIII-encoding nucleotide sequence is efficiently packaged in a recombinant AAV (rAAV) vector. Administration of the rAAV vector carrying a B-domain deleted human factor VIII (BDD human factor VIII) under the control of a liver-preferred enhancer element to mice resulted in long-term expression (>14 months) of B-domain deleted human factor VIII by the liver and therapeutic levels of B-domain deleted human factor VIII protein (˜27% of normal) in the plasma of treated animals. Accordingly, the present invention provides novel reagents and methods for the treatment of hemophilia A using a rAAV vector for gene delivery.

A rAAV vector is an AAV virus particle that carries a heterologous (i.e., foreign) gene in its genome. rAAV vectors require at least one of the 145 base terminal repeats in cis of the 4679 wild type bases to generate virus. All other viral sequences are dispensable and may be supplied in trans (Muzyczka, (1992) Curr. Topics Microbiol. Immunol 158:97). Typically, rAAV vectors will only retain the minimal terminal repeat sequences so as to maximize the size of the transgene that can be efficiently packaged by the vector.

As used herein, “infection” or “transduction” of a cell by AAV means that the AAV enters the cell to establish a latent or active infection. See, e.g., Fields et al., Virology, Volume 2, Chapter 69 (3d ed., Lippincott-Raven Publishers). In embodiments of the invention in which the AAV is administered to a subject, it is preferred that the AAV integrates into the genome and establishes a latent infection. However, such integration is not required for expression of a transgene carried by a rAAV vector as the vector can persist stably as an episome in transduced cells.

Except as otherwise indicated, standard methods may be used for the construction of rAAV vectors, helper vectors, and cells according to the present invention. Such techniques are known to those skilled in the art (see, e.g., Sambrook et al. (1989) Molecular Cloning: A Laboratory Manual (2d ed., Cold Spring Harbor Laboratory Press, Plainview, N.Y.); Aububel et al. (1995) Current Protocols in Molecular Biology (Green Publishing Associates, Inc. and John Wiley & Sons, Inc., NY).

A. rAAV Vectors Encoding B-domain Deleted Factor VIII.

The present invention provides a construct encoding a biologically-active B-domain deleted factor VIIII that can be efficiently packaged, delivered, and expressed using a rAAV vector. In some embodiments, an AAV ITR comprised in the rAAV vector drives expression of the B-domain deleted factor VIII nucleotide sequence without an additional promoter. The rAAV vectors of the invention include at least one enhancer and at least one promoter to promote expression. rAAV/factor VIII vectors according to the present invention may be produced in sufficient titers to permit administration to cells and subjects for the production of the encoded B-domain deleted factor VIII protein or for therapeutic treatment (for veterinary or medical uses, e.g., to enhance blood coagulation or to treat hemophilia A).

These results are unexpected in light of the known packaging limitations of AAV vectors. These limitations place constraints on the size of the heterologous nucleotide sequences and/or expression control elements that may be efficiently packaged by the AAV capsid (see, e.g., Russell et al. (1999) Blood 94:864; Chuah et al. (1998) Critical Review in Oncology/Hematology 28:153).

The full-length factor VIII gene is 186 kb in length and encodes a 9029 nucleotide mRNA. A cDNA encoding the full-length factor VIII would greatly exceed the packaging capacity of rAAV vectors. It has been found that the B domain is not necessary for factor VIII function. Deletion of the sequences encoding the B-domain produces an approximately 4.4 to 4.6 kb cDNA B-domain deleted factor VIII. The art teaches that even this smaller construct could not be efficiently packaged and expressed using a rAAV vector because of the challenge of adding adequate expression control elements (e.g., promoters, enhancers, poly(A) site) for high-level expression without exceeding the size limitations for high titer production in AAV (Russell et al. ((1999) Blood 94:864, at page 868, col. 1, para. 2).

Accordingly, it was quite surprising that the present inventors achieved an efficient packaging of the recombinant vector such that a high titer rAAV/B-domain deleted human factor VIII stock was achieved. Particularly in view of the fact that the rAAV vector used a transgene expression cassette that was 109% of wild-type (5084 bp). Moreover, this B-domain deleted human factor VIII vector is expressed long-term and at high levels by hepatocytes in vivo and produces therapeutic levels of B-domain deleted human factor VIII protein in plasma of treated animals.

As indicated the present invention provides rAAV vectors carrying a heterologous nucleotide sequence encoding a biologically active B-domain deleted factor VIII. The nucleotide sequence encoding the B-domain deleted factor VIII may be from any species, including avian and mammalian species. Preferably, the B-domain deleted factor VIII is mammalian (e.g., mouse, rat, lagomorph, feline, canine, bovine, porcine, ovine, caprine, equine, simian, human, and the like), more preferably the B-domain deleted factor VIII is a human B-domain deleted factor VIII. As a further alternative, the B-domain deleted factor VIII may an inter-species hybrid, as described below. The nucleotide sequences may also be a synthetic sequence. Variants and fragments of the B-domain deleted factor VIII sequence are also encompassed, so long as they retain factor VIII biological activity.

The biologically active B-domain deleted factor VIII coding sequences must be sufficiently small so that they can be packaged by AAV. It is preferred that the size of the B-domain deleted factor VIII transgene construct be about 4.8 kb or shorter, more preferably about 4.7 kb or shorter, yet more preferably about 4.6 kb or shorter, yet more preferably about 4.5 kb or shorter, still more preferably less than about 4.4 kb or shorter.

Alternatively stated, it is preferred that the B-domain deleted factor VIII transgene cassette (i.e., including ITRs and other expression control elements) is about 5.2 kb or shorter, about 5.1 kb or shorter, about 5.0 kb or shorter, about 4.9 kb or shorter, 4.8 kb or shorter, about 4.7 kb or shorter, about 4.5 kb or shorter, or about 4.4 kb or shorter. The B-domain deleted factor VIII transgene cassette is of a size that can be efficiently packaged to produce rAAV stocks.

The B-domain deleted factor VIII transgene may be truncated and/or deleted to achieve the size described above. Any truncation and/or deletion known in the art may be employed as long as the expressed B-domain deleted factor VIII protein retains sufficient biological activity (e.g., coagulation). By “sufficient biological activity”, is intended that the B-domain deleted factor VIII possesses enough activity to be of use in vitro and/or in vivo. Preferably, the expressed truncated and/or deleted B-domain deleted factor VIII retains at least about 25%, about 50%, about 75%, about 85%, about 90%, about 95%, about 98%, about 99% or more of the biological activity of the native factor VIII protein. Assays for determining factor VIII biological activity are well known in the art and include those assays described herein. See also Practor and Rapaport (1961) Blood 72:335 for a description of the one-stage clotting assay for determining specific activity of factor VIII. Factor VIII activity may also be measured in a chromogenic assay (Kabi Coatest; Kabi Vitrurus, Stockholm, Sweden).

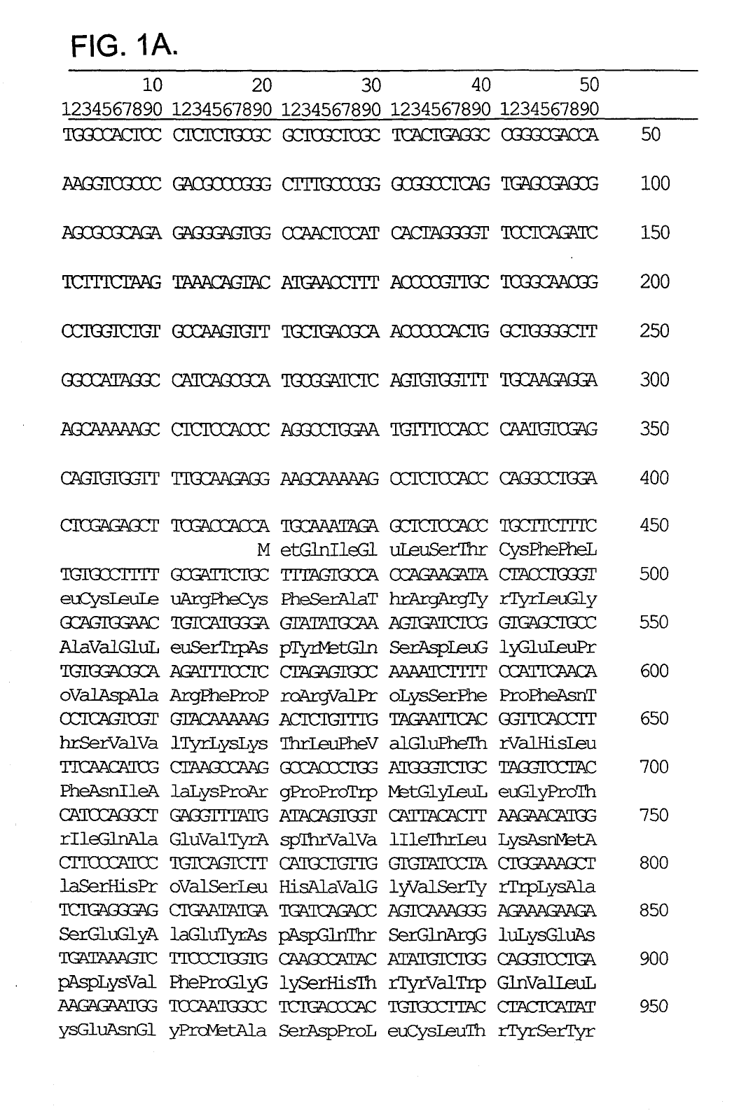

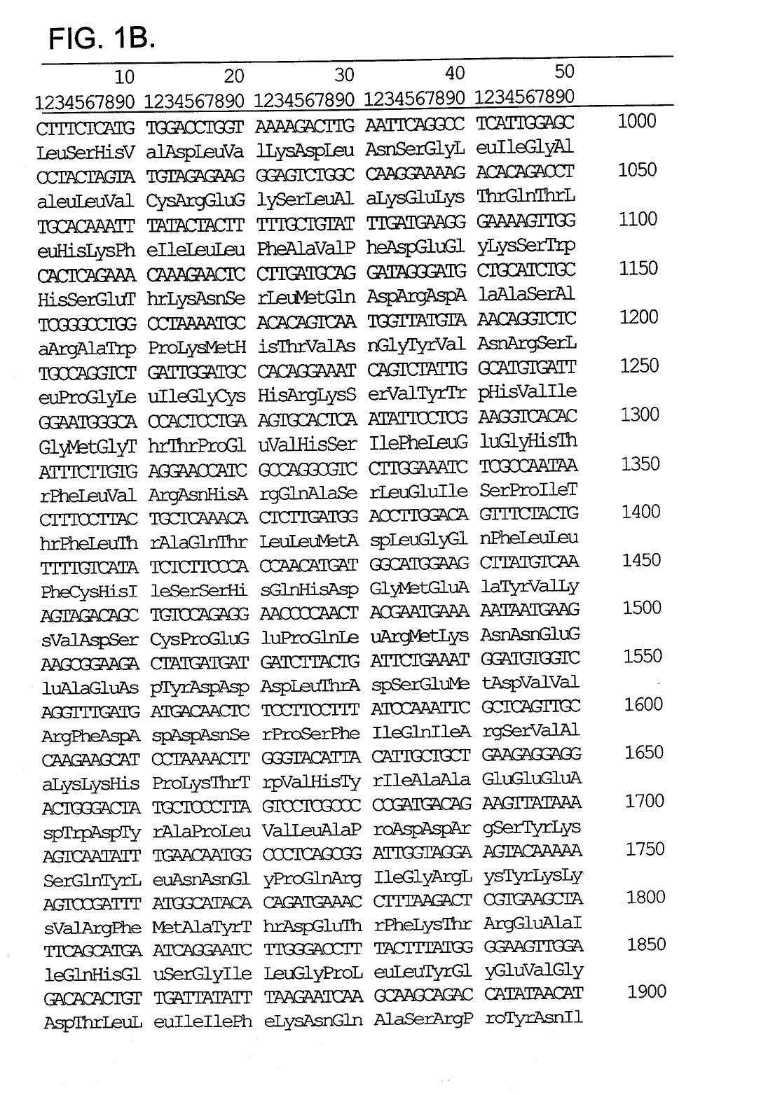

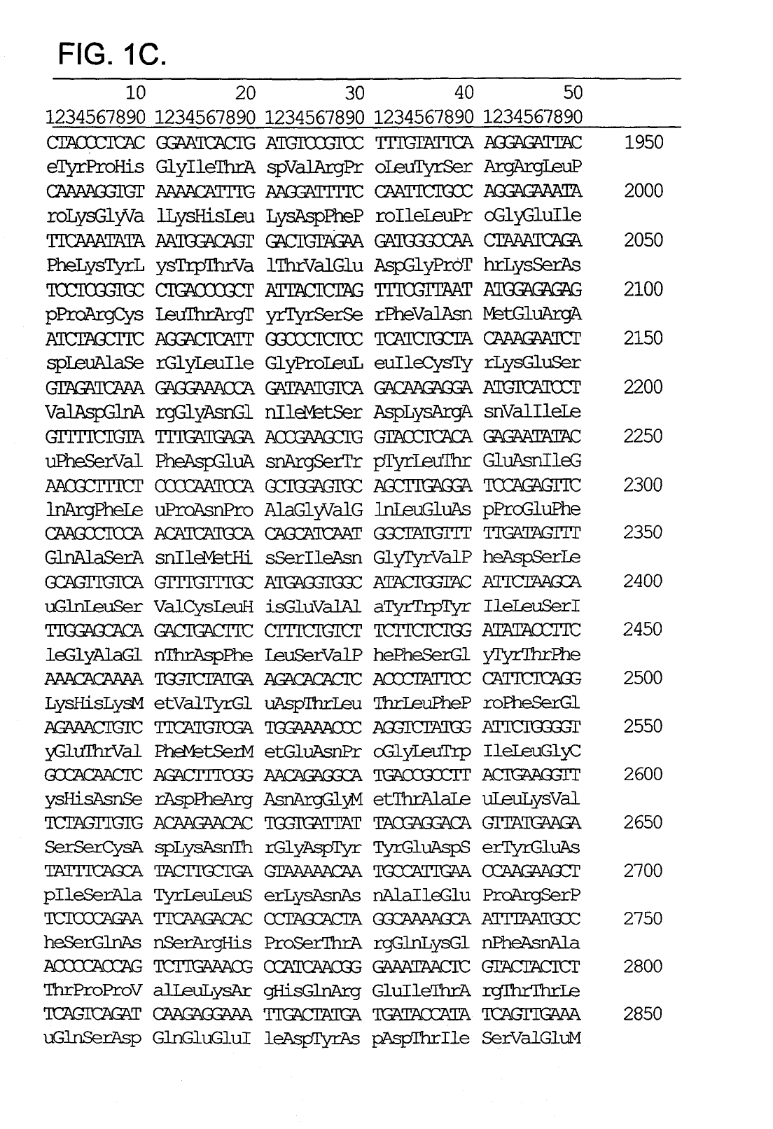

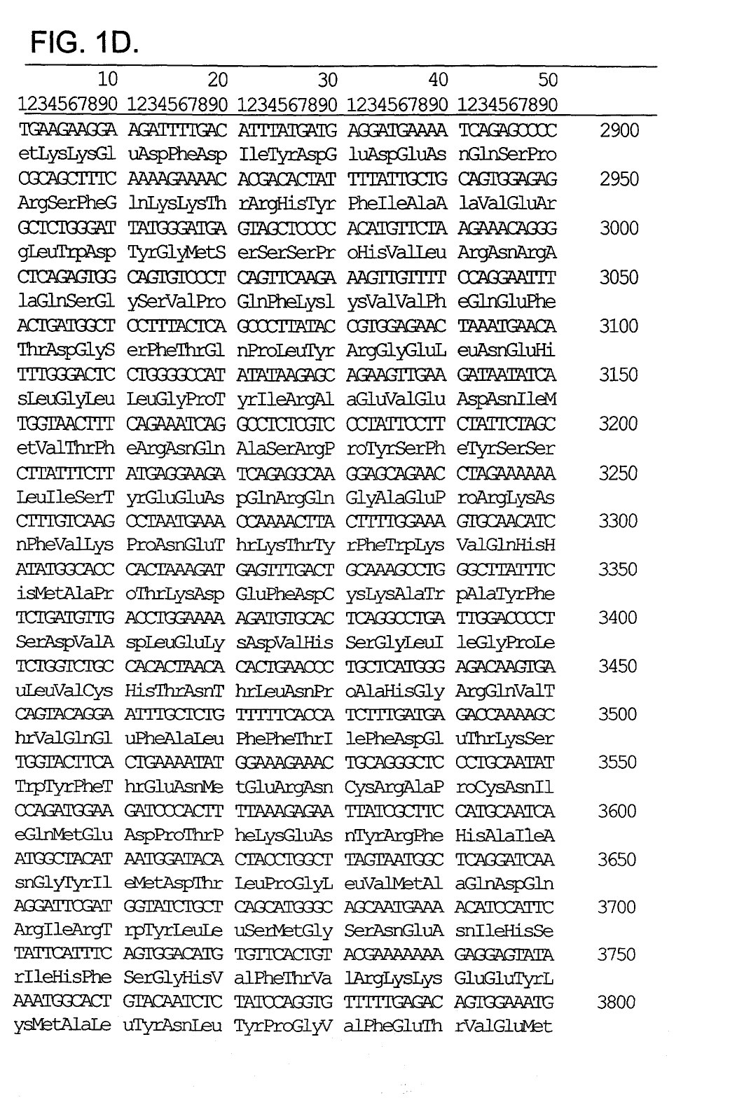

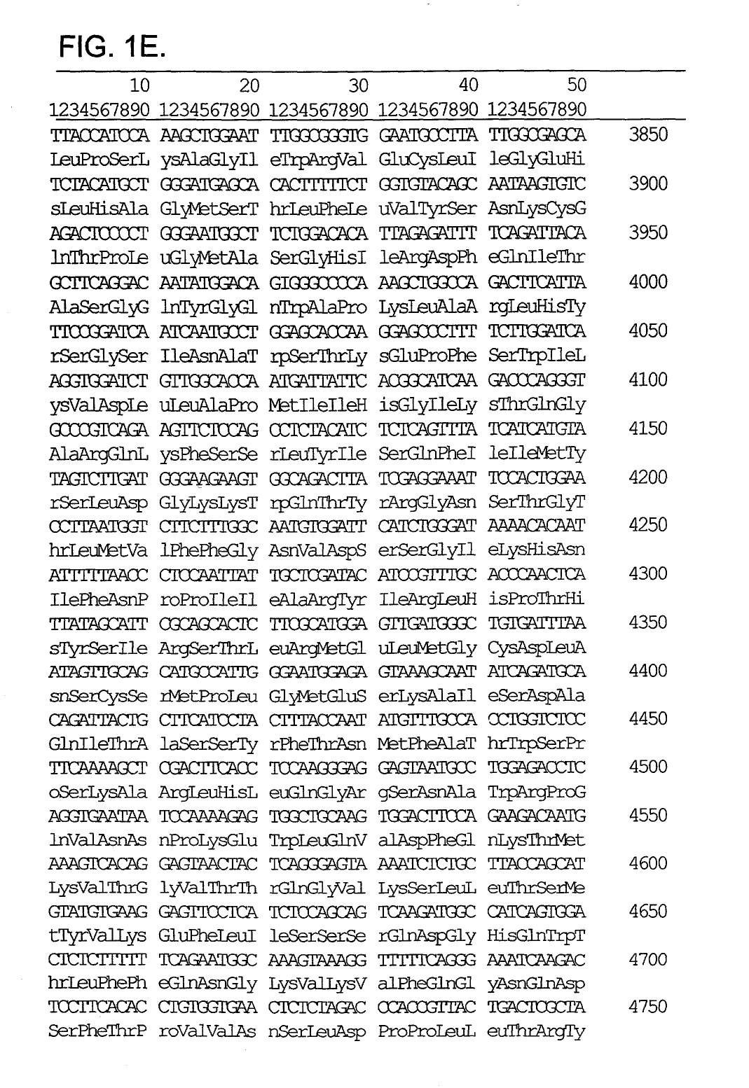

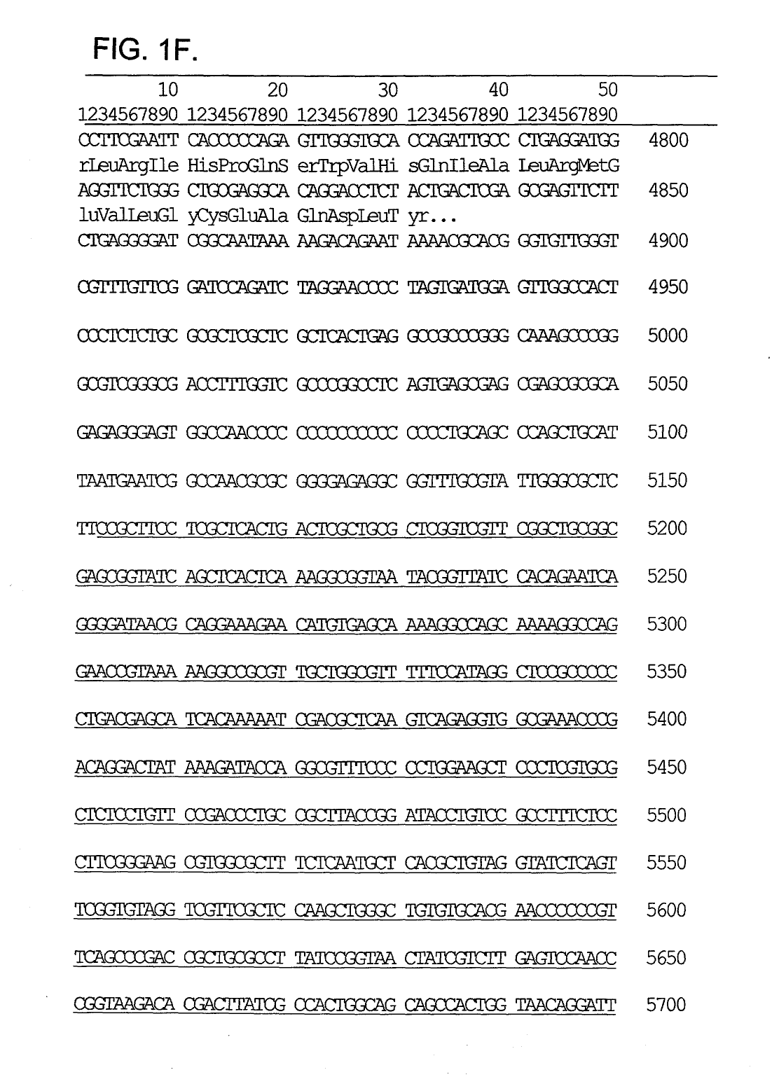

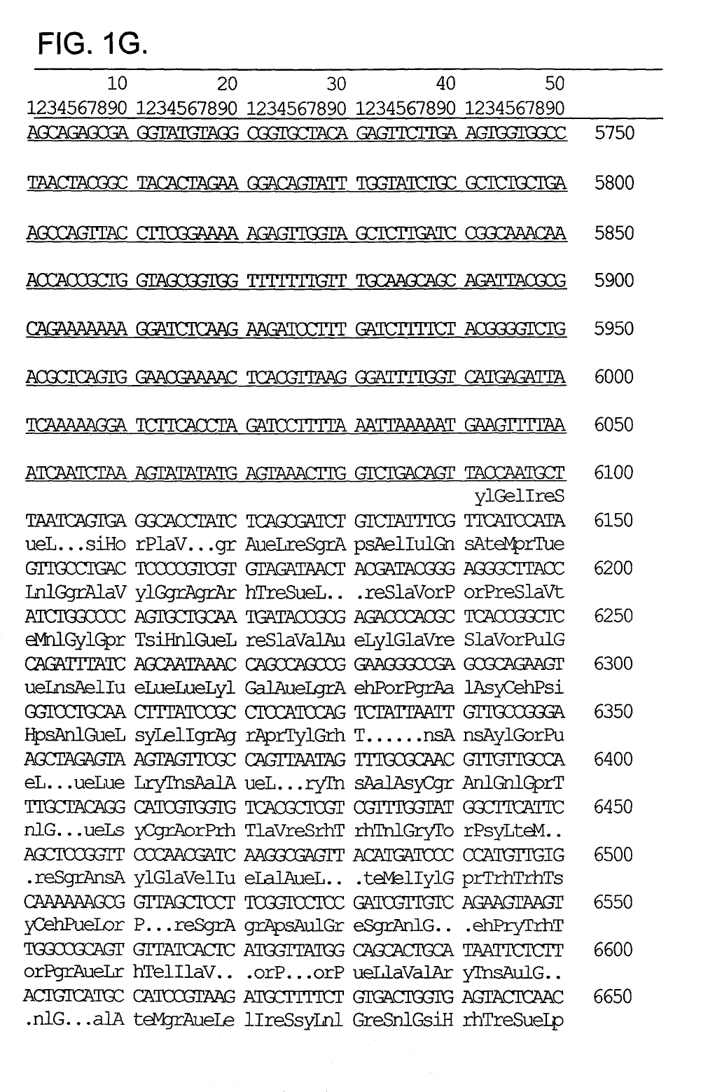

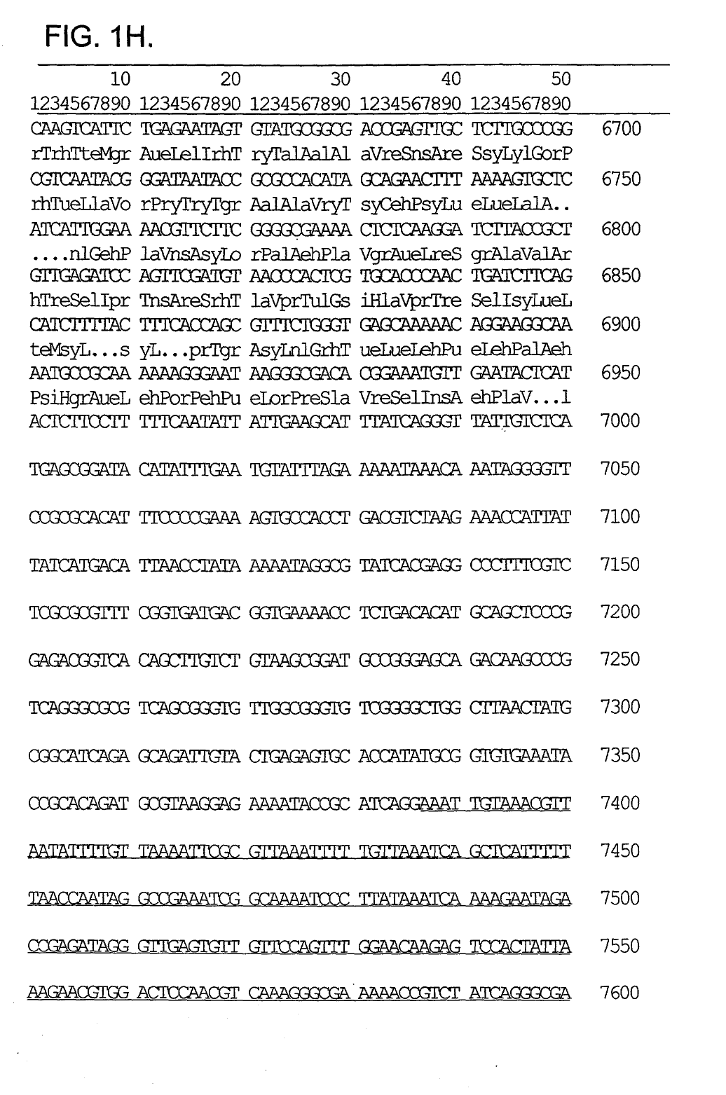

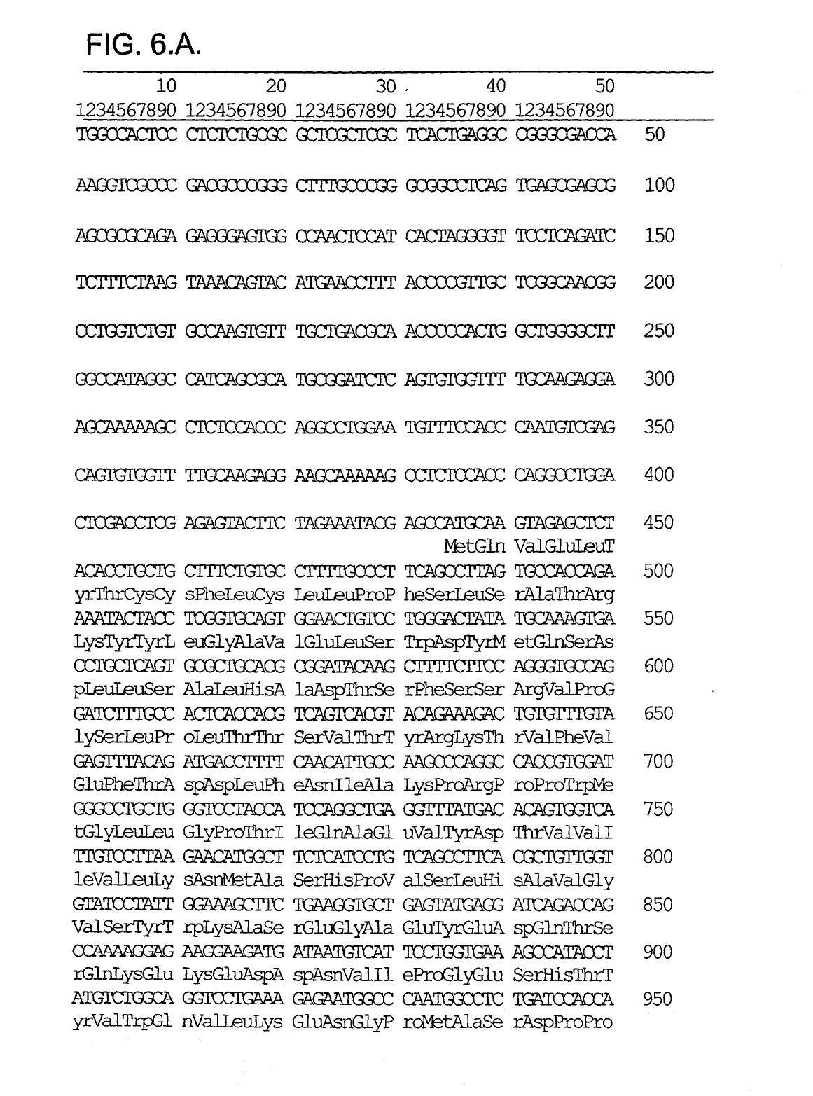

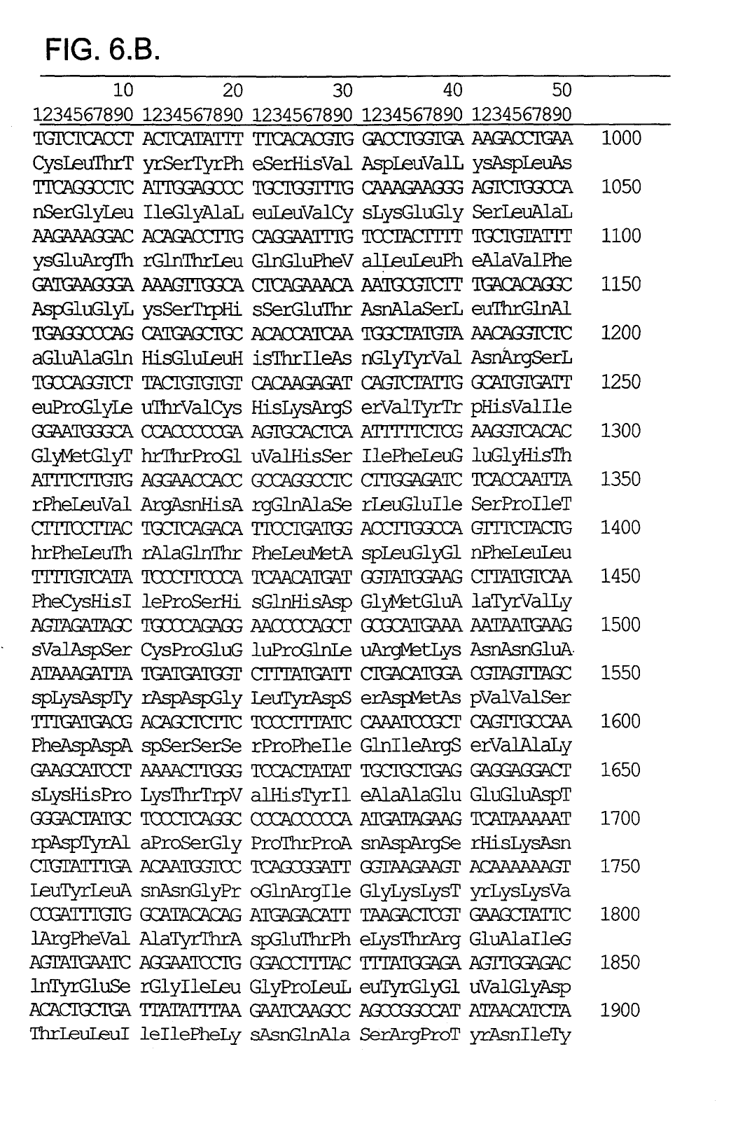

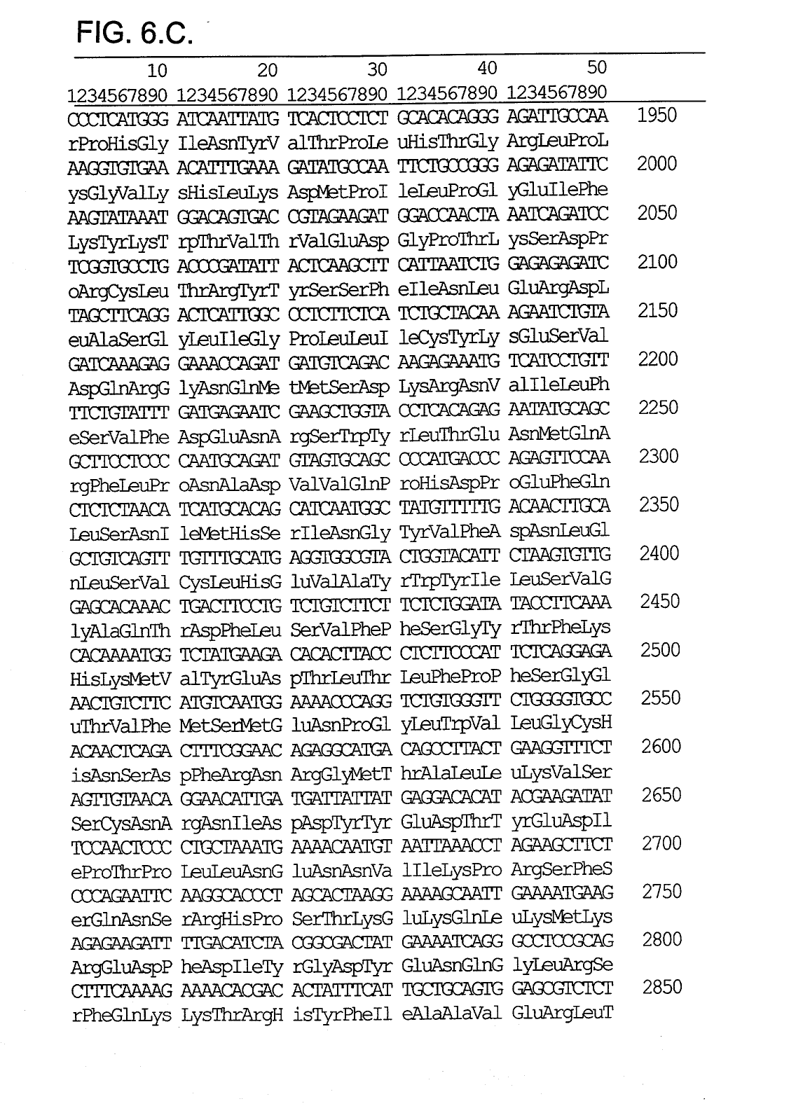

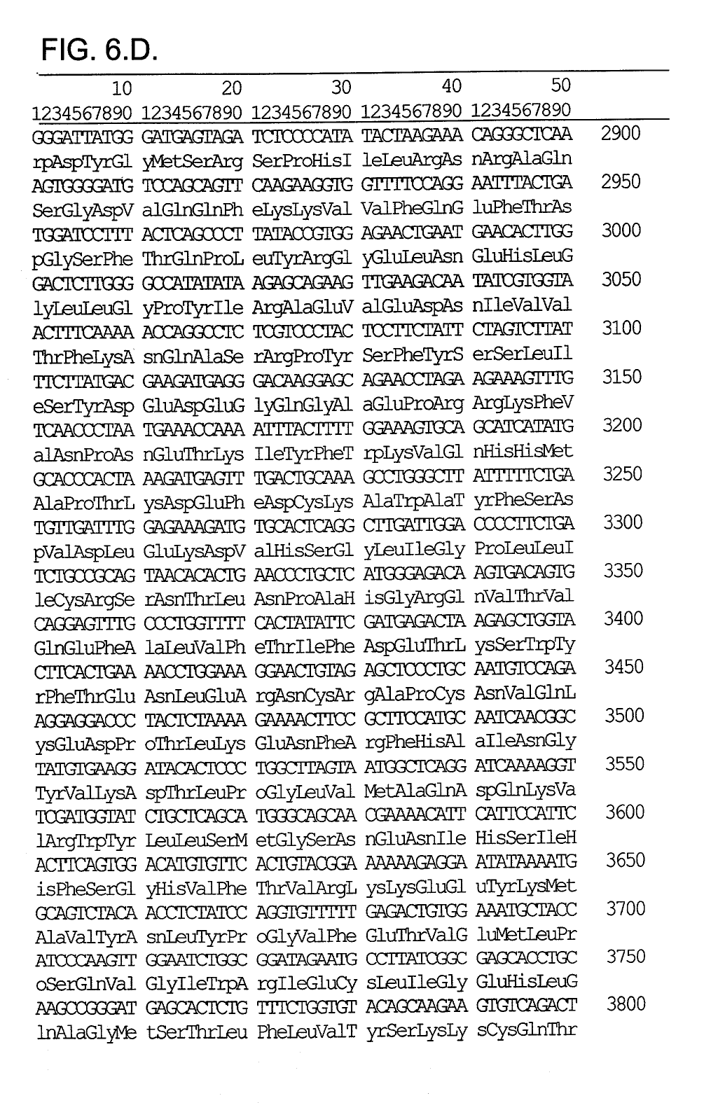

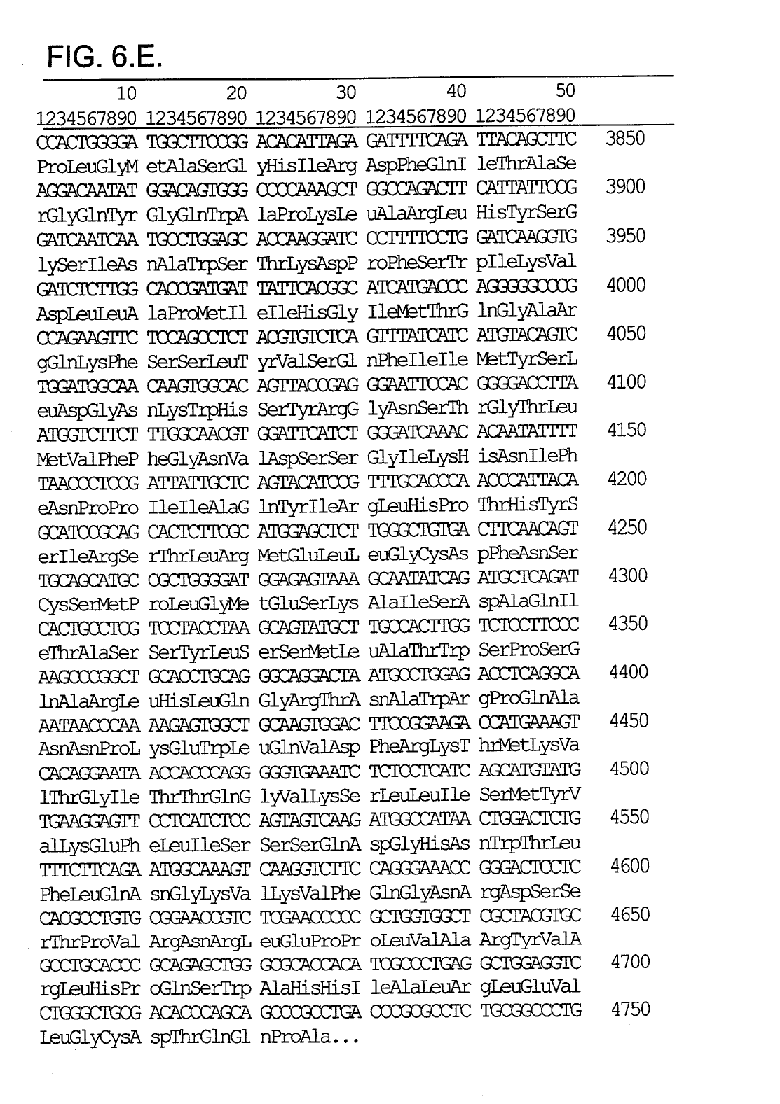

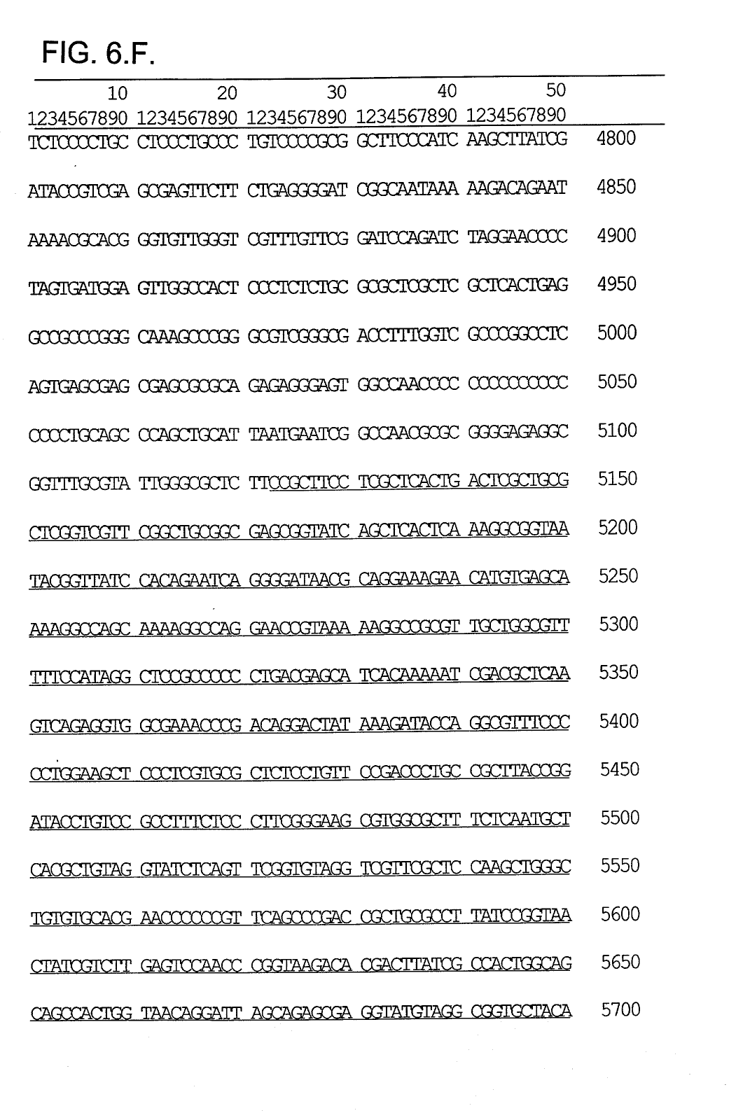

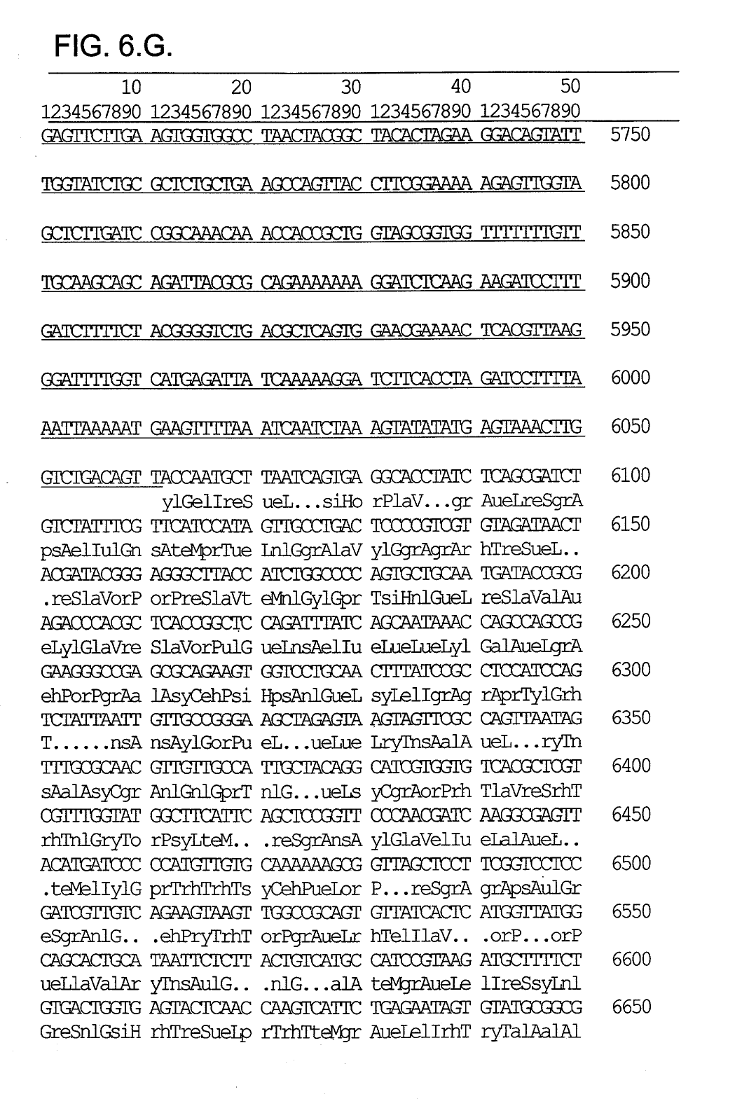

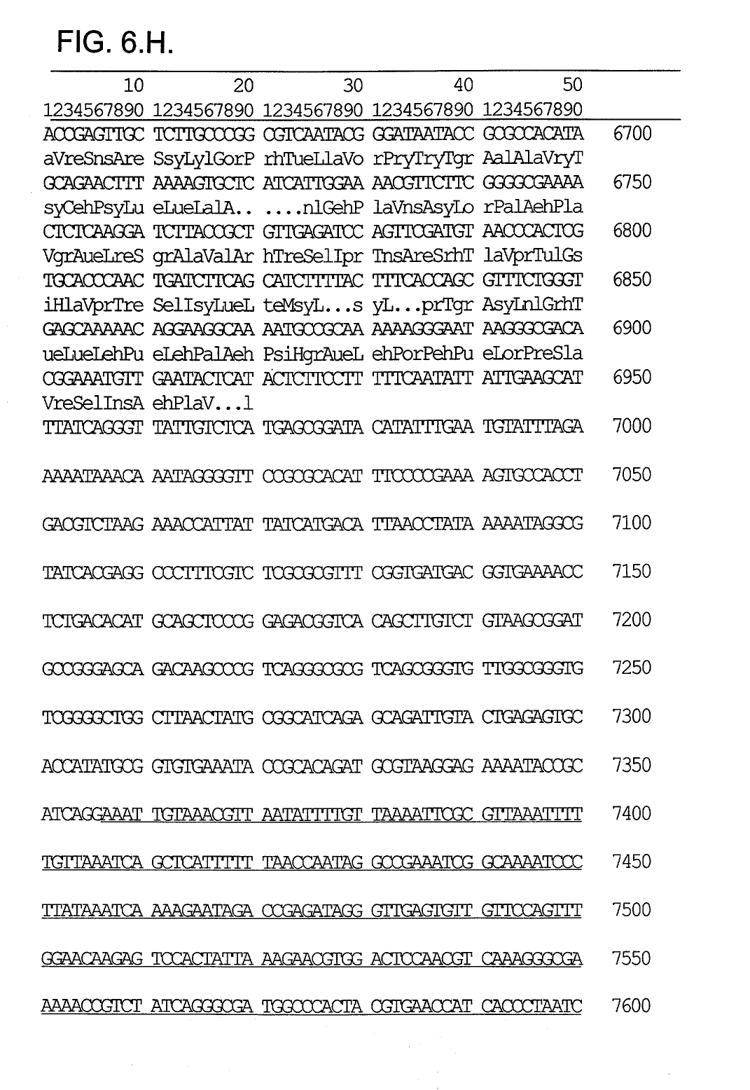

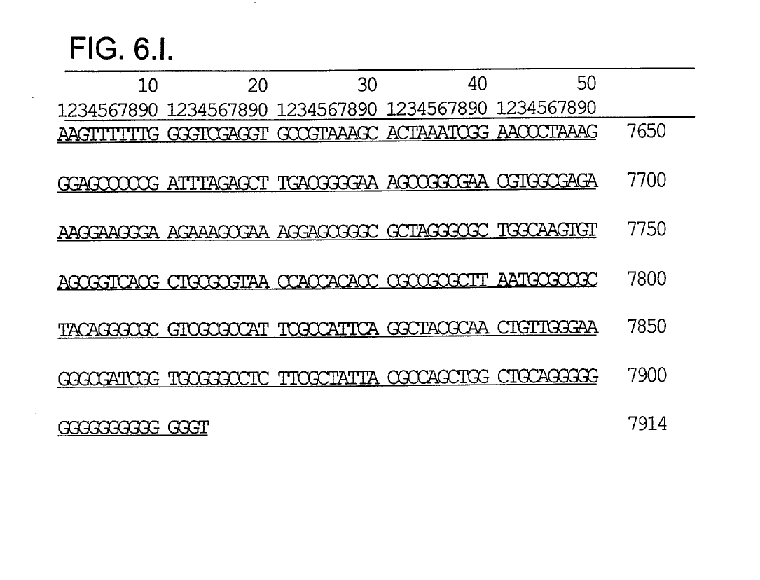

In preferred embodiments, the B-domain deleted factor VIII constructs of the present invention will contain deletions in the nucleotide sequences encoding the B-domain. Nucleotide sequences encoding portions or all of the B-domain can be deleted to minimize transgene size. The constructs of the invention may retain some nucleotide sequences from the B-domain deleted region as a result of the cloning strategy employed. The amino acid sequence of one human B-domain deleted factor VIII is provided herein in FIG. 1 and in SEQ ID NO:2, and is encoded by nucleotides 419 to 4835 of the nucleotide sequence shown in this figure and in SEQ ID NO:1. B-domain-deleted factor VIII mutant has deleted residues 760 through 1639 (factor VIII 760-1639) (Pittman et al. (1993) Blood 11:2925. Other B-domain deleted factor VIII are known in the art and include those encoded by the factor VIIIΔ756-1679 and factor VIIIΔ761-1639 constructs described by Gnatenko et al. (1999) Br. J. Haemotology 104:27, and the factor VIII 746-1639 construct described by Ill et al. (1997) Blood Coagulation and Fibrinolylsis 8:523. See also U.S. Pat. No. 5,910,481, where several B-domain deleted mutants are described. The invention further provides a canine construct having the amino acid sequence set forth in FIG. 6 and SEQ ID NO:4. The canine B-domain deleted factor VIII (B-domain deleted-canine factor VIII) mutant protein is encoded by nucleotides 428-4790 of the nucleotide sequence set forth in FIG. 6 (SEQ ID NO:3). This construct also has residues 760-1639 deleted from the B-domain. Variants and fragments of the B-domain deleted human factor VIII and B-domain deleted canine factor VIII nucleotide sequences are also encompassed by the present invention.

In some embodiments, the expression cassette and/or the nucleotide sequence encoding B-domain deleted factor VIII has been modified to increase, for example, the efficiency of transcription and/or translation of the B-domain deleted factor VIII transgene. Such modifications are known in the art and are described, for example, in Ill et al. (1997) Blood Coagul. Fibrinolysis 8(suppl. 2):S23-S30, herein incorporated by reference.

In other embodiments of the invention, the nucleotide sequence encoding the biologically active B-domain deleted factor VIII is substantially identical to the sequence given as about nucleotides 419 to 4835 of FIG. 1 (SEQ ID NO:1) or to the sequence given as about nucleotides 428-4790 of FIG. 6 (SEQ ID NO:3), and encodes a biologically-active or therapeutically effective B-domain deleted factor VIII . This definition is intended to include natural allelic variations in the factor VIII gene. B-domain deleted factor VIII according to this embodiment may come from any species of origin, or may be a hybrid, each as described above. As used herein, nucleotide sequences that are “substantially identical” are at least 75%, and more preferably at least 80%, 85%, 90%, 95%, or even 99% identical or more, that is they share at least 75%, and more preferably at least 80%, 85%, 90%, 95%, or even 99% identity or more with the disclosed sequences. Sequence identity may be determined by methods described elsewhere herein.

High stringency hybridization conditions which will permit substantially identical nucleotide sequences to hybridize are well known in the art. For example, hybridization of homologous nucleotide sequences to the sequence given as about nucleotides 419-4835 of the sequence shown in FIG. 1 (SEQ ID NO:1) or to the sequence given as about nucleotides 428-4790 of the sequence shown in FIG. 6 (SEQ ID NO:3) maybe carried out in 25% formamide, 5×SSC, 5×Denhardt's solution, with 100 μg/ml of single stranded DNA and 5% dextran sulfate at 42° C. for 4, 8, or 12 hours, with wash conditions of 25% formamide, 5×SSC, 0.1% SDS at 42° C. for 15 minutes, to allow hybridization of sequences of about 60% homology. More stringent conditions are represented by a wash stringency of 0.3M NaCl, 0.03 M sodium citrate, 0.1% SDS at 60° or even 70° C. using a standard in situ hybridization assay. See Sambrook et al. (1989) Molecular Cloning: A Laboratory Manual (2d ed., Cold Spring Harbor Laboratory Press, Plainview, N.Y.).

Those skilled in the art will appreciate that the B-domain deleted factor VIII construct may contain other modifications as long as the expressed B-domain deleted factor VIII retains sufficient biological activity (as described above). For example, the B-domain deleted factor VIII protein may be modified to enhance biological activity, extend the half-life of the protein, or reduce antigenic responses in recipients being administered the B-domain deleted factor VIII (see, e.g., Kaufman et al. (1998) Haemophilia 4:370, the disclosure of which is incorporated herein in its entirety). As a further alternative, the B-domain deleted factor VIII may be an inter-species hybrid. For example, human/porcine hybrids of factor VIII have been described by U.S. Pat. No. 5,583,209 (the disclosure of which is incorporated herein in its entirety). Likewise, domain swaps between factor V and factor VIII have produced hybrids with increased half-life and/or biological activity.

Suitable biologically active variants of a native or naturally occurring protein or polypeptide of interest can be fragments, analogues, and derivatives of that polypeptide. By “fragment” is intended a polypeptide consisting of only a part of the intact polypeptide sequence and structure, and can be a C-terminal deletion or N-terminal deletion of the native polypeptide. By “analogue” is intended an analogue of either the native polypeptide or of a fragment of the native polypeptide, where the analogue comprises a native polypeptide sequence and structure having one or more amino acid substitutions, insertions, or deletions. By “derivative” is intended any suitable modification of the native protein or polypeptide of interest, of a fragment of the native protein or polypeptide, or of their respective analogues, such as glycosylation, phosphorylation, or other addition of foreign moieties, so long as the desired biological activity of the native protein or polypeptide is retained. Methods for making such fragments, analogues, and derivatives are generally available in the art.

For example, amino acid sequence variants of the protein or polypeptide can be prepared by mutations in the cloned DNA sequence encoding the native protein or polypeptide of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (Cold Spring Harbor, New York); U.S. Pat. No. 4,873,192; and the references cited therein; herein incorporated by reference. Guidance as to appropriate amino acid substitutions that do not affect biological activity of the polypeptide of interest may be found in the model of Dayhoff et al. (1978) in Atlas of Protein Sequence and Structure (Natl. Biomed. Res. Found., Washington, D.C.), herein incorporated by reference. Conservative substitutions, such as exchanging one amino acid with another having similar properties, may be preferred. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr.

In constructing variants of the protein or polypeptide of interest, modifications are made such that variants continue to possess the desired activity. Obviously, any mutations made in the DNA encoding the variant protein or polypeptide must not place the sequence out of reading frame and preferably will not create complementary regions that could produce secondary mRNA structure. See EP Patent Application Publication No. 75,444.

Biologically active variants of a protein or polypeptide of interest will generally have at least 70%, preferably at least 80%, more preferably about 90% to 95% or more, and most preferably about 98% or more amino acid sequence identity to the amino acid sequence of the reference polypeptide molecule, which serves as the basis for comparison. A biologically active variant of a native polypeptide of interest may differ from the native polypeptide by as few as 1-15 amino acids, as few as 1-10, such as 6-10, as few as 5, as few as 4, 3, 2, or even 1 amino acid residue. By “sequence identity” is intended the same amino acid residues are found within the variant protein or polypeptide and the protein or polypeptide molecule that serves as a reference when a specified, contiguous segment of the amino acid sequence of the variant is aligned and compared to the amino acid sequence of the reference molecule. The percentage sequence identity between two amino acid sequences is calculated by determining the number of positions at which the identical amino acid residue occurs in both sequences to yield the number of matched positions, dividing the number of matched positions by the total number of positions in the segment undergoing comparison to the reference molecule, and multiplying the result by 100 to yield the percentage of sequence identity.

For purposes of optimal alignment of the two sequences, the contiguous segment of the amino acid sequence of the variant may have additional amino acid residues or deleted amino acid residues with respect to the amino acid sequence of the reference molecule. The contiguous segment used for comparison to the reference amino acid sequence will comprise at least twenty (20) contiguous amino acid residues, and may be 30, 40, 50, 100, or more residues. Corrections for increased sequence identity associated with inclusion of gaps in the variant's amino acid sequence can be made by assigning gap penalties. Methods of sequence alignment are well known in the art for both amino acid sequences and for the nucleotide sequences encoding amino acid sequences.

Thus, the determination of percent identity between any two sequences can be accomplished using a mathematical algorithm. One preferred, non-limiting example of a mathematical algorithm utilized for the comparison of sequences is the algorithm of Myers and Miller (1988) CABIOS 4:11-17. Such an algorithm is utilized in the ALIGN program (version 2.0), which is part of the GCG sequence alignment software package. A PAM120 weight residue table, a gap length penalty of 12, and a gap penalty of 4 can be used with the ALIGN program when comparing amino acid sequences. Another preferred, nonlimiting example of a mathematical algorithm for use in comparing two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to a nucleotide sequence encoding the polypeptide of interest. BLAST protein searches can be performed with the XBLAST program, score=50, wordlength=3, to obtain amino acid sequences homologous to the polypeptide of interest. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Also see the ALIGN program (Dayhoff (1978) in Atlas of Protein Sequence and Structure 5:Suppl. 3 (National Biomedical Research Foundation, Washington, D.C.) and programs in the Wisconsin Sequence Analysis Package, Version 8 (available from Genetics Computer Group, Madison, Wis.), for example, the GAP program, where default parameters of the programs are utilized.

When considering percentage of amino acid sequence identity, some amino acid residue positions may differ as a result of conservative amino acid substitutions, which do not affect properties of protein function. In these instances, percent sequence identity may be adjusted upwards to account for the similarity in conservatively substituted amino acids. Such adjustments are well known in the art. See, for example, Myers and Miller (1988) Computer Applic. Biol. Sci. 4:11-17.

Those skilled in the art will appreciate that a variety of expression control elements (e.g., promoter and/or transcription factor binding sites and/or enhancers) may be operably linked with the heterologous nucleotide sequence encoding the B-domain deleted factor VIII depending on the level and tissue-preferred expression desired. As noted above, generally, the expression control element will comprise at least one enhancer element. However, it is recognized that a promoter or promoter element may also be included in the cassette.

Selection of promoters or promoter elements is based in part on size. Small or minimal promoters may be preferred due to the packaging size constraints imposed by the AAV vector.

A variety of promoters may be used in the rAAV vectors of the invention, provided the size constraints noted above are met. These include, but are not limited to, the herpes simplex virus thymidine kinase or thymidylate synthase promoters (Merrill (1989) Proc. Natl. Acad. Sci. USA 86:4987, Deng et al. (1989) Mol. Cell. Biol. 9:4079), the hepatitis B virus core promoter (see, for example, Kramvis and Kew (1999) J. Viral. Hepat. 6:415-427), the human U1 snRNA promoter (see, for example, Asselbergs and Pronk (1993) Mol. Biol. Rep. 17:101-114), the mouse minimal albumin promoter with proximal elements (see, for example Pinkert et al. (1987) Genes Dev. 1:268-276), the promoters described in the PCT publication WO09920773 (herein incorporated by reference), the minimal cytomegalovirus major immediate early promoter, the early and late SV40 promoters, the adenovirus major late promoter, the alpha- or beta-interferon promoters, event or tissue preferred promoters, etc. Promoters may be chosen so as to potently drive expression or to produce relatively weak expression, as desired.

In one embodiment, rAAV vectors of the invention comprise B-domain deleted factor VIII coding sequences under the transcriptional control of a liver-preferred enhancer element, and an event-specific promoter, such that upon activation of the event-specific promoter the gene of interest encoded by the B-domain deleted factor VIII nucleic acid molecule is expressed. As used herein, an “event-specific promoter” is a promoter that is activated upon under certain cellular conditions. Numerous event-specific promoters may be utilized within the context of the present invention, including, without limitation, promoters which are activated by cellular proliferation (or are otherwise cell-cycle dependent) such as the thymidine kinase or thymidylate synthase promoters, or the transferrin receptor promoter, which will be transcriptionally active primarily in rapidly proliferating cells (such as hematopoietic cells) that contain factors capable of activating transcription from these promoters preferentially to express and secrete B-domain deleted factor VIII into the blood stream; promoters such as the alpha- or beta-interferon promoters, which are activated when a cell is infected by a virus (Fan and Maniatis (1989) EMBO J. 8:101; Goodbourn et al. (1986) Cell 45:601); and promoters that are activated by the presence of hormones, e.g., estrogen response promoters. See Toohey et al. (1986) Mol. Cell. Biol, 6:4526.

In another embodiment, rAAV vectors of the invention comprise the B-domain deleted factor VIII gene under the transcriptional control of a liver-preferred enhancer and a liver-preferred promoter, such that upon activation of the liver-preferred promoter, the B-domain deleted factor VIII gene is expressed. Representative examples of such liver-preferred promoters include, but are not limited to Phospho-Enol-Pyruvate Carboxy-Kinase (“PEPCK”) (Hatzoglou et al.(1988) J. Biol. Chem. 263:17798; Benvenisty et al. (1989) Proc. Natl. Acad. Sci. USA 86:1118; Vaulont et al. (1989) Mol. Cell. Biol. 6:4409), the alcohol dehydrogenase promoter (Felder (1989) Proc. Natl. Acad. Sci. USA 86:5903), and the albumin promoter and the alphafetoprotein promoter (Feuerman et al. (1989) Mol. Cell. Biol. 9:4204; Camper and Tilghman (1989) Genes Develop. 3:537).

The present invention also encompasses embodiments in which the rAAV vectors contain promoter elements that are binding sites for specific transcription factors These promoter elements are referred to herein as “transcription factor binding sites.” The transcription factors that bind these sites may be ubiquitous or tissue-preferred. Non-limiting examples of binding sites for ubiquitous transcription factors include the TATA box (TATAAAA), which binds TFIID; the CAAT box (GGCCAATCT), which binds CTF/NF; the GC box (GGGCGG), which binds SP1, and the ATF box (GTGACGT), which binds ATF. Non-limiting examples of tissue-preferred transcription factor binding sites include the liver-preferred CAAT box binding sites for C/EBP proteins (optimal palindrome GATTGCGCAATC; set forth in SEQ ID NO:5); the binding sites for HNF1, HNF3, and HNF4 (see, for example, Costa and Grayson (1991) Nucleci Acids Res. 19:4139-4145); and the binding site for TGT3 (see, for example, Chiang et al. (1992) Biochim. Biophys. Acta 1132:337-339).

In some embodiments of the invention, the expression control element comprises an enhancer for liver-preferred expression of the transgene. Non-limiting examples of such enhancers encompassed by the present invention include the α1 microglobulin/bikunin enhancer (see, for example, Rouet et al. (1992) J. Biol. Chem. 267:20765029773), the hepatitis B virus EnhI (e.g. nucleotides 150-278 of FIG. 1 or SEQ ID NO:1 and Guo et al. (1991) J. Virol. 65:6686-6692) and EnhII (Gustin et al. (1993) Virology 193(2):653-60) enhancers, the human albumin E_{1.7} and E₆ enhancers (Hayashi et al (1992) J. Biol. Chem. 267:14580-14585), and the human cytomegalovirus immediate early gene enhancer (Boshart et al. (1985) Cell 41:521-530).

While any expression control element(s) known in the art may be employed, those skilled in the art will understand that the expression control element(s) employed will preferably comply with the size constraints described for AAV vectors.

In addition, the rAAV vectors of the invention may contain polyadenylation signals operably linked with the heterologous nucleic acid sequence(s) to be delivered to the target cell. These polyadenylation sequences preferably conform to the size limitations described above. Preferred polyadenylation comprise less than about 100 bp. In one embodiment, the poladenylation signal is a synthetic polyadenylation signal (see, for example WO 09920773, herein incorporated by reference).

In one embodiment of the invention, the B-domain deleted factor VIII transgene cassette is as shown in FIG. 1 (SEQ ID NO:1). This construct includes the left and right AAV terminal repeats and, in the 5′ to 3′ direction, the hepatitis B virus EnhI enhancer (nt 150-278), spacer sequence (nt 279-399), a B-domain deleted human factor VIII coding region (nt 419-4835), and the TK polyadenylation sequence (nt 4840-4914).

B. Methods of Producing rAAV Stocks.

There are at least three desirable features of an rAAV virus preparation for use in gene transfer. First, it is preferred that the rAAV virus should be generated at titers sufficiently high to transduce an effective proportion of cells in the target tissue. A high number of rAAV infectious units are typically required for gene transfer in vivo. For example, some treatments may require in excess of about 10⁸ particles, about 10⁹ particles, about 10¹⁰ particles, about 10¹¹ particles, about 10¹² particles, about 10¹³ particles, about 10¹⁴ particles, about 10¹⁵ particles. Second, it is preferred that the rAAV virus preparations should be essentially free of replication-competent AAV (i.e., phenotypically wild-type AAV which can be replicated in the presence of helper virus or helper virus functions). Third, it is preferred that the rAAV virus preparation as a whole be essentially free of other viruses (such as a helper virus used in AAV production) as well as helper virus and cellular proteins, and other components such as lipids and carbohydrates, so as to minimize or eliminate any risk of generating an immune response in the context of gene transfer. This latter point is especially significant in the context of AAV because AAV is a “helper-dependent” virus that requires co-infection with a helper virus (typically adenovirus) or other provision of helper virus functions in order to be effectively replicated and packaged during the process of AAV production; and, moreover, as described above, adenovirus has been observed to generate a host immune response in the context of gene transfer applications (see, e.g., Le et al. (1997); Byrnes et al. (1995) Neuroscience 66:1015; McCoy et al. (1995) Human Gene Therapy 6:1553; and Barr et al. (1995) Gene Therapy 2:151).

In order to replicate and package the rAAV vector, the missing functions are complemented with a packaging gene, or a plurality thereof, which together encode the necessary functions for the various missing rep and/or cap gene products. The packaging genes or gene cassettes are preferably not flanked by AAV ITRs and preferably do not share any substantial homology with the rAAV genome.

The rAAV vector construct and complementary packaging gene constructs can be implemented in this invention in a number of different forms. Viral particles, plasmids, and stably transformed host cells can all be used to introduce such constructs into the packaging cell, either transiently or stably.

A variety of different genetically altered cells can thus be used in the context of this invention. By way of illustration, a mammalian host cell may be used with at least one intact copy of a stably integrated rAAV vector. An AAV packaging plasmid comprising at least an AAV rep gene operably linked to a promoter can be used to supply replication functions (as described in U.S. Pat. No. 5,658,776). Alternatively, a stable mammalian cell line with an AAV rep gene operably linked to a promoter can be used to supply replication functions (see, e.g., Trempe et al., U.S. Pat. No. 5,837,484; Burstein et al., WO 98/27207; and Johnson et al., U.S. Pat. No. 5,658,785). The AAV cap gene, providing the encapsidation proteins as described above, can be provided together with an AAV rep gene or separately (see, e.g., the above-referenced applications and patents as well as Allen et al. (WO 96/17947). Other combinations are possible.

As is described in the art, and illustrated in the references cited above and in Examples below, genetic material can be introduced into cells (such as mammalian “producer” cells for the production of rAAV) using any of a variety of means to transform or transduce such cells. By way of illustration, such techniques include, but are not limited to, transfection with bacterial plasmids, infection with viral vectors, electroporation, calcium phosphate precipitation, and introduction using any of a variety of lipid-based compositions (a process often referred to as “lipofection”). Methods and compositions for performing these techniques have been described in the art and are widely available.

Selection of suitably altered cells may be conducted by any technique in the art. For example, the polynucleotide sequences used to alter the cell may be introduced simultaneously with or operably linked to one or more detectable or selectable markers as is known in the art. By way of illustration, one can employ a drug resistance gene as a selectable marker. Drug resistant cells can then be picked and grown, and then tested for expression of the desired sequence (i.e., a product of the heterologous polynucleotide). Testing for acquisition, localization and/or maintenance of an introduced polynucleotide can be performed using DNA hybridization-based techniques (such as Southern blotting and other procedures as known in the art). Testing for expression can be readily performed by Northern analysis of RNA extracted from the genetically altered cells, or by indirect immunofluorescence for the corresponding gene product. Testing and confirmation of packaging capabilities and efficiencies can be obtained by introducing to the cell the remaining functional components of AAV and a helper virus, to test for production of AAV particles. Where a cell is inheritably altered with a plurality of polynucleotide constructs, it is generally more convenient (though not essential) to introduce them to the cell separately, and validate each step seriatim. References describing such techniques include those cited herein.

In one approach to packaging rAAV vectors in an AAV particle, the rAAV vector sequence (i.e., the sequence flanked by AAV ITRs), and the AAV packaging genes to be provided in trans, are introduced into the host cell in separate bacterial plasmids. Examples of this approach are described in Ratschin et al. (1984) Mol. Cell. Biol. 4:2072; Hermonat et al.(1984) Proc. Natl. Acad. Sci. USA 81:6466; Tratschin et al. (1985) Mol. Cell. Biol. 5:3251; McLaughlin et aL (988)J. Virol. 62:1963; Lebkowski et al. (188) Mol. Cell. Biol. 7:349; Samulski et al. (989) J. Virol. 63:3822-3828; and Flotte et al. (1992) Am. J. Respir. Cell. Mol. Biol. 7:349.

A second approach is to provide either the rAAV vector sequence, or the AAV packaging genes, in the form of an episomal plasmid in a mammalian cell used for AAV replication. See, for example, U.S. Pat. No. 5,173,414.

A third approach is to provide either the rAAV vector sequence or the AAV packaging genes, or both, stably integrated into the genome of the mammalian cell used for replication.

One exemplary technique of this third approach is outlined in international patent application WO 95/13365 (Targeted Genetics Corporation and Johns Hopkins University) and corresponding U.S. Pat. No. 5,658,776 (by Flotte et al.). This example uses a mammalian cell with at least one intact copy of a stably integrated rAAV vector, wherein the vector comprises an AAV ITR and a transcription promoter operably linked to a target polynucleotide, but wherein the expression of rep is limiting in the cell. In a preferred embodiment, an AAV packaging plasmid comprising the rep gene operably linked to a heterologous promoter is introduced into the cell, and then the cell is incubated under conditions that allow replication and packaging of the rAAV vector sequence into particles.

Another approach is outlined in Trempe et al., U.S. Pat. No. 5,837,484. This example uses a stable mammalian cell line with an AAV rep gene operably linked to a heterologous promoter so as to be capable of expressing functional Rep protein. In various preferred embodiments, the AAV cap gene can be provided stably as well or can be introduced transiently (e.g. on a plasmid). An rAAV vector can also be introduced stably or transiently.

Another approach is outlined in patent application WO 96/17947 (Targeted Genetics Corporation). This example uses a mammalian cell which comprises a stably integrated AAV cap gene, and a stably integrated AAV rep gene operably linked to a helper virus-inducible heterologous promoter. A plasmid comprising the rAAV vector sequence is also introduced into the cells (either stably or transiently). The packaging of rAAV vector into particles is then initiated by introduction of the helper virus.

Methods for achieving high titers of rAAV virus preparations that are substantially free of contaminating virus and/or viral or cellular proteins are outlined by Atkinson et al. in WO 99/11764. Techniques described therein can be employed for the large-scale production of rAAV viral particle preparations.

These various examples address the issue of producing rAAV viral particles at sufficiently high titer, minimizing recombination between rAAV vector and sequences encoding packaging components, reducing or avoiding the potential difficulties associated with the expression of the AAV rep gene in mammalian cell line (since the Rep proteins can not only limit their own expression but can also affect cellular metabolism) and producing rAAV virus preparations that are substantially free of contaminating virus and/or viral or cellular protein.

Packaging of an AAV vector into viral particles relies on the presence of a suitable helper virus for AAV or the provision of helper virus functions. Helper viruses capable of supporting AAV replication are exemplified by adenovirus, but include other viruses such as herpes viruses (including, but not limited to, HSV1, cytomegalovirus and HHV-6) and pox virus (particularly vaccinia). Any such virus may be used.

Frequently, the helper virus will be an adenovirus of a type and subgroup that can infect the intended host cell. Human adenovirus of subgroup C, particularly serotypes 1, 2, 3, 4, 5, 6, and 7, are commonly used. Serotype 5 is generally preferred.

The features and growth patterns of adenovirus are known in the art. See, for example, Horowitz, “Adenoviridae and their replication”, pp 771-816 in “Fundamental Virology”, Fields et al., eds. The packaged adenovirus genome is a linear DNA molecule, linked through adenovirus ITRs at the left- and right-hand termini through a terminal protein complex to form a circle. Control and encoding regions for early, intermediate, and late components overlap within the genome. Early region genes are implicated in replication of the adenovirus genome, and are grouped depending on their location into the E1, E2, E3, and E4 regions.

Although not essential, in principle it is desirable that the helper virus strain be defective for replication in the subject ultimately to receive the genetic therapy. Thus, any residual helper virus present in an rAAV virus preparation will be replication-incompetent. Adenoviruses from which the E1A or both the E1A and the E3 region have been removed are not infectious for most human cells. They can be replicated in a permissive cell line (e.g., the human 293 cell line) which is capable of complementing the missing activity. Regions of adenovirus that appear to be associated with helper function, as well as regions that do not, have been identified and described in the art (see, e.g., P. Colosi et al., WO97/17458, and references cited therein).

For example, as described in Atkinson et al. (WO 99/11764), a “conditionally-sensitive” helper virus can also be employed to provide helper virus activity. Such a helper virus strain must minimally have the property of being able to support AAV replication in a host cell under at least one set of conditions where it itself does not undergo efficient genomic replication. Where helper virus activity is supplied as intact virus particles, it is also generally necessary that the virus be capable of replication in a host cell under a second set of conditions. The first set of conditions will differ from the second set of conditions by a readily controllable feature, such as the presence or absence of a required cofactor (such as a cation), the presence or absence of an inhibitory drug, or a shift in an environmental condition such as temperature. Most conveniently, the difference between the two conditions is temperature, and such a conditionally-sensitive virus is thus referred to as a temperature-sensitive helper virus.

Helper virus may be prepared in any cell that is permissive for viral replication. For adenovirus, preferred cells include 293 cells and HeLa cells. It is preferable to employ culture techniques that permit an increase in seeding density. 293 cells and HeLa cell variants are available that have been adapted to suspension culture. HeLa is preferable for reasons of cell growth, viability and morphology in suspension. These cells can be grown at sufficient density (2×10⁶ per ml) to make up for the lower replication rate of the temperature-sensitive adenovirus strain. Once established, cells are infected with the virus and cultured at the permissive temperature for a sufficient period; generally 3-7 days and typically about 5 days.

Examples of methods useful for helper virus preparation, isolation and concentration can be found in Atkinson et al. (WO 99/11764).

Several criteria influence selection of cells for use in producing rAAV particles as described herein. As an initial matter, the cell must be permissive for replication and packaging of the rAAV vector when using the selected helper virus. However, since most mammalian cells can be productively infected by AAV, and many can also be infected by helper viruses such as adenovirus, it is clear that a large variety of mammalian cells and cell lines effectively satisfy these criteria. Among these, the more preferred cells and cell lines are those that can be easily grown in culture so as to facilitate large-scale production of rAAV virus preparations. Again, however, many such cells effectively satisfy this criterion. Where large-scale production is desired, the choice of production method will also influence the selection of the host cell. For example, as described in more detail in Atkinson et al. (WO 99/11764) and in the art, some production techniques and culture vessels or chambers are designed for growth of adherent or attached cells, whereas others are designed for growth of cells in suspension. In the latter case, the host cell would thus preferably be adapted or adaptable to growth in suspension. However, even in the case of cells and cell lines that are regarded as adherent or anchorage-dependent, it is possible to derive suspension-adapted variants of an anchorage-dependent parental line by serially selecting for cells capable of growth in suspension. See, for example, Atkinson et al. (WO 99/11764).

Ultimately, the helper virus, the rAAV vector sequence, and all AAV sequences needed for replication and packaging must be present in the same cell. Where one or more AAV packaging genes are provided separately from the vector, a host cell is provided that comprises: (i) one or more AAV packaging genes, wherein each said AAV packaging gene encodes an AAV replication or encapsidation protein; (ii) a heterologous polynucleotide introduced into said host cell using an rAAV vector, wherein said rAAV vector comprises said heterologous polynucleotide flanked by at least one AAV ITR and is deficient in said AAV packaging gene(s); and (iii) a helper virus or sequences encoding the requisite helper virus functions. It should be noted, however, that one or more of these elements may be combined on a single replicon.

The helper virus is preferably introduced into the cell culture at a level sufficient to infect most of the cells in culture, but can otherwise be kept to a minimum in order to limit the amount of helper virus present in the resulting preparation. A multiplicity of infection or “MOI” of 1-100 may be used, but an MOI of 5-10 is typically adequate.

Similarly, if the rAAV vector and/or packaging genes are transiently introduced into the packaging cell (as opposed to being stably introduced), they are preferably introduced at a level sufficient to genetically alter most of the cells in culture. Amounts generally required are of the order of 10 μg per 10⁶ cells, if supplied as a bacterial plasmid; or 10⁸ particles per 10⁵ cells, if supplied as an AAV particle. Determination of an optimal amount is an exercise of routine titration that is within the ordinary skill of the artisan.

These elements can be introduced into the cell, either simultaneously, or sequentially in any order. Where the cell is inheritably altered by any of the elements, the cell can be selected and allowed to proliferate before introducing the next element.

In one preferred example, the helper virus is introduced last into the cell to rescue and package a resident rAAV vector. The cell will generally already be supplemented to the extent necessary with AAV packaging genes. Preferably, either the rAAV vector or the packaging genes, and more preferably both are stably integrated into the cell. It is readily appreciated that other combinations are possible. Such combinations are included within the scope of the invention.

Once the host cell is provided with the requisite elements, the cell is cultured under conditions that are permissive for the replication AAV, to allow replication and packaging of the rAAV vector. Culture time is preferably adjusted to correspond to peak production levels, and is typically 3-6 days. rAAV particles are then collected, and isolated from the cells used to prepare them.

Optionally, rAAV virus preparations can be further processed to enrich for rAAV particles, deplete helper virus particles, or otherwise render them suitable for administration to a subject. See Atkinson et al. for exemplary techniques (WO 99/11764). Purification techniques can include isopynic gradient centrifugation, and chromatographic techniques. Reduction of infectious helper virus activity can include inactivation by heat treatment or by pH treatment as is known in the art. Other processes can include concentration, filtration, diafiltration, or mixing with a suitable buffer or pharmaceutical excipient. Preparations can be divided into unit dose and multi dose aliquots for distribution, which will retain the essential characteristics of the batch, such as the homogeneity of antigenic and genetic content, and the relative proportion of contaminating helper virus.

Various methods for the determination of the infectious titer of a viral preparation are known in the art. For example, one method for titer determination is a high-throughput titering assay as provided by Atkinson et al. (WO 99/11764). Virus titers determined by this rapid and quantitative method closely correspond to the titers determined by more classical techniques. In addition, however, this high-throughput method allows for the concurrent processing and analysis of many viral replication reactions and thus has many others uses, including for example the screening of cell lines permissive or non-permissive for viral replication and infectivity.

A preferred method for providing helper functions through infectious adenovirus employs a non-infectious adenovirus miniplasmid that carries all of the helper genes required for efficient AAV production (Ferrari et al. (1997) Nature Med. 3:1295; Xiao et al. (1998) J. Virology 72:2224). The rAAV titers obtained with adenovirus miniplasmids are forty-fold higher than those obtained with conventional methods of wild-type adenovirus infection (Xiao et al. (1998) J. Virology 72:2224). This approach obviates the need to perform co-transfections with adenovirus (Holscher et al. (1994) J. Virology 68:7169; Clark et al. (1995) Hum. Gene Ther. 6:1329; Trempe and Yang (1993), in, Fifth Parvovirus Workshop (Crystal River, Fla.).

Other methods of producing rAAV stocks have been described, including but not limited to, methods that split the rep and cap genes onto separate expression cassettes to prevent the generation of replication-competent AAV (Allen et al. (1997) J. Virol. 71:6816), and methods employing packaging cell lines (Gao et al. (1998) Human Gene Therapy 9:2353; Inoue et al. (1998) J. Virol. 72:7024).

The present invention provides methods of producing a high titer rAAV vector stocks carrying the B-domain deleted factor VIII transgenes and B-domain deleted factor VIII expression cassettes of the invention. These results are surprising as prior attempts to produce rAAV/factor VIII have failed to generate adequate titers of virus for in vivo administration.

The inventive methods of producing high titer rAAV/B-domain deleted factor VIII stock involves infecting a packaging cell with a rAAV vector carrying a heterologous nucleotide sequence encoding a B-domain deleted factor VIII, as described above. The rAAV vector is replicated and packaged by the packaging cell, and the rAAV particles are collected to form an AAV stock. This stock has a titer of at least about 10⁴, about 10⁵, about 10⁶, about 10⁷, about 10⁸, about 10⁹, about 10¹⁰, about 10¹¹, about 10¹², or about 10¹³ particles per milliter.

Preferred packaging cells for producing rAAV stocks are known in the art and include packaging cells for producing rAAV by methods involving adenovirus helper virus or adenovirus miniplasmids, including but not limited to, 293 cells (see, e.g., Samulski et al. (1989) J. Virology 63:3822; Ferrari et al. (1997) Nature Med. 3:1295; Xiao et al. (1998) J. Virology 72:2224). Other rAAV packaging cells include those described by Gao et al. (1998) Human Gene Therapy 9:2353 and Inoue et al. (1998) J. Virol. 72:7024.

C. Gene Transfer Technology.

The methods of the present invention provide a means for delivering heterologous nucleotide sequences into a broad range of host cells, including dividing and non-dividing cells both in vitro (e.g., to produce factor VIII protein or for ex vivo gene therapy) and in vivo. The vectors, methods, and pharmaceutical formulations of the present invention are additionally useful in a method of administering a protein or peptide to a subject in need thereof, or a method of treatment or otherwise. In this manner, the protein or peptide may thus be produced in vivo in the subject. The subject may be in need of the protein or peptide because the subject has a deficiency of the protein or peptide, or because the production of the protein or peptide in the subject may impart some therapeutic effect, as a method of treatment or otherwise, and as explained further below.

In general, the present invention can be employed to deliver any heterologous nucleotide sequence encoding a biologically-active B-domain deleted factor VIII that can be packaged by a rAAV vector, as described above. The heterologous nucleotide sequence encoding the B-domain deleted factor VIII gene may be administered to a subject to achieve a therapeutic effect. For example, the heterologous nucleotide sequence encoding the B-domain deleted factor VIII may be administered to enhance (e.g., improve, increase, augment) blood coagulation.

D. Subjects, Pharmaceutical Formulations, Vaccines and Modes of Administration.

The present invention finds use in veterinary and medical applications. Suitable subjects include both avians and mammals, with mammals being preferred. The term “avian” as used herein includes, but is not limited to, chickens, ducks, geese, quail, turkeys and pheasants. The term “mammal” as used herein includes, but is not limited to, humans, bovines, ovines, caprines, equines, felines, canines, lagomorphs, etc. Human subjects are most preferred. Human subjects include neonates, infants, juveniles, and adults.

In particular embodiments, the present invention provides a pharmaceutical composition comprising a rAAV particle of the invention in a pharmaceutically acceptable carrier or other medicinal agents, pharmaceutical agents, carriers, adjuvants, diluents, etc. For injection, the carrier will typically be a liquid. For other methods of administration, the carrier may be either solid or liquid, such as sterile, pyrogen-free water or sterile pyrogen-free phosphate-buffered saline solution. For inhalation administration, the carrier will be respirable, and will preferably be in solid or liquid particulate form. As an injection medium, it is preferred to use water that contains the additives usual for injection solutions, such as stabilizing agents, salts or saline, and/or buffers.

By “pharmaceutically acceptable” is intended a material that is not biologically or otherwise undesirable, i.e., the material may be administered to a subject along with the viral vector without causing any undesirable biological effects. Thus, such a pharmaceutical composition can be used, for example, in transfection of a cell ex vivo or in administering a viral particle directly to a subject.

The present invention further provides a method of delivering a heterologous nucleotide sequence encoding B-domain deleted factor VIII to a cell. For in vitro methods, the virus can be administered to the cell by standard viral transduction methods, as are known in the art. Preferably, the virus particles are added to the cells at the appropriate multiplicity of infection according to standard transduction methods appropriate for the particular target cells. Titers of virus to administer can vary, depending upon the target cell type and the particular virus vector, and can be determined by those of skill in the art without undue experimentation. Alternatively, administration of a rAAV vector of the present invention can be accomplished by any other means known in the art.

The cell to be administered the inventive virus vector can be of any type, including but not limited to neural cells (including cells of the peripheral and central nervous systems, in particular, brain cells), retinal cells, epithelial cells (e.g., gut and respiratory), muscle cells, pancreatic cells (including islet cells), hepatic cells, myocardial cells, bone cells (e.g., bone marrow stem cells), hematopoietic stem cells, spleen cells, fibroblasts, endothelial cells, germ cells, and the like. Moreover, the cells can be from any species of origin, as indicated above.

In particular embodiments of the invention, cells are removed from a subject, the rAAV vector is introduced therein, and the cells are then replaced back into the subject. Methods of removing cells from a subject for treatment ex vivo, followed by introduction back into the subject are known in the art. Alternatively, the rAAV vector is introduced into cells from another subject or from cultured cells to express the B-domain deleted factor VIII therein, and the cells are administered to a subject in need of factor VIII therapy. Suitable cells for ex vivo gene therapy include, but are not limited to, liver cells, neural cells (including cells of the central and peripheral nervous systems, in particular, brain cells), pancreas cells, spleen cells, fibroblasts (e.g., skin fibroblasts), keratinocytes, endothelial cells, epithelial cells, myoblasts, hematopoietic stem cells, and bone marrow stromal cells.

A further aspect of the invention is a method of treating subjects in vivo with the inventive virus particles. Administration of the rAAV particles of the present invention to a human subject or an animal in need thereof can be by any means known in the art for administering virus vectors. A “therapeutically effective” amount as used herein is an amount of the rAAV/B-domain deleted factor VIII vector that is sufficient to alleviate (e.g., mitigate, decrease, reduce) at least one of the symptoms associated with factor VIII deficiency (e.g., blood coagulation). It is not necessary that the administration of the B-domain deleted factor VIII eliminate the symptoms of Factor VIII deficiency, as long as the benefits outweigh the detriments of B-domain deleted factor VIII administration.

The normal range of factor VIII in human plasma is approximately 100-200 ng/ml. Normal blood clotting is seen with plasma factor VIII levels that are as low as 5% of normal. Therapeutic effects may be observed with as little as 1% of normal plasma factor VIII levels (Nilsson et al. (1992) J. Int. Med. 232:25-32; Lofgvist et al. (1997) J. Int. Med. 241:395-400; Petrini et al. (1991) Am. J. Ped. Hem. Onc.13:280-287; and Hematology-Principles and Practice, 3rd ed. (2000) Hoffman, R; ed., pages 1884-1885). Administration of a rAAV/B-domain deleted factor VIII vector of the invention to a subject preferably results in plasma factor VIII levels that are at least about 1% of normal, more preferably at least about 5% of normal, still more preferably at least about 10% of normal, yet more preferably at least about 20% of normal, still yet more preferably at least about 25% of normal factor VIII levels.

In particularly preferred embodiments of the invention, the nucleotide sequence of interest is delivered to the liver of the subject. Administration to the liver can be achieved by any method known in the art, including, but not limited to intravenous administration, intraportal administration, intrabiliary administration, intra-arterial administration, and direct injection into the liver parenchyma.

Accordingly, a further aspect of the present invention is a method of treating a subject with factor VIII deficiency, including hemophilia A. As used herein, a factor VIII deficiency may be due to a defective protein or lack of protein. Preferably, the subject is a human subject. According to this method, the subject is administered n an amount of a rAAV/factor VIII vector sufficient to produce a biologically effective amount of factor VIII to one or more tissues. Preferably, the tissue is brain, pancreas, spleen, liver, reticulum endothelial system (RES), lymphoid, or muscle, or bone marrow/stromal cells, most preferably, the liver.

In preferred embodiments, the rAAV vector is administered to the liver. Preferably, the cells (e.g., liver cells) are infected by the rAAV/B-domain deleted factor VIII vector, express the B-domain deleted factor VIII protein, and secrete the protein into the circulatory system in a therapeutically effective amount as defined above. It is not necessary that the symptoms of factor VIII deficiency be eliminated, as long as the benefits outweigh the detriments of administering the factor VIII.

Exemplary modes of administration include oral, rectal, transmucosal, topical, transdermal, inhalation, parenteral (e.g., intravenous, subcutaneous, intradermal, intramuscular, and intraarticular) administration, and the like, as well as direct tissue or organ injection, alternatively, intratrahecal, direct intramuscular, intraventricular, intravenous, intraperitoneal, intranasal, or intraocular injections. Injectables can be prepared in conventional forms, either as liquid solutions or suspensions, solid forms suitable for solution or suspension in liquid prior to injection, or as emulsions. Alternatively, one may administer the virus in a local rather than systemic manner, for example, in a depot or sustained-release formulation.

In preferred embodiments, the inventive rAAV vectors are administered by intravenous administration, more preferably, by intravenous administration to the liver (as described below).

Dosages will depend upon the mode of administration, the severity of the disease or condition to be treated, the individual subject's condition, the particular virus vector, and the gene to be delivered, and the species of the subject, the size and weight of the subject, and can be determined in a routine manner. Exemplary doses for achieving therapeutically effective amounts in the circulatory system are about 10⁸, about 10⁹, about 10¹⁰, about 10¹¹, about 10¹², about 10¹³, about 10¹⁴, about 10¹⁵ infectious units, depending upon the level of transgene produced, the activity of the protein, etc.

The invention will now be illustrated with reference to certain examples which are included herein for the purposes of illustration only, and which are not intended to be limiting of the invention.

### EXAMPLE 1

Vector Constructs

rAAV plasmids expressing human B-domain deleted factor VIII or enhanced green fluorescent protein (EGFP) were constructed. Briefly, pmt2LA (Pittman et al. (1993) Blood 81:2925; gift from Dr. D. Pittman, Genetics Institute, Cambridge, Mass.) was amplified by PCR to generate a 4435 bp fragment encoding full sequence of B-domain deleted-human factor VIII. The 4435 bp B-domain deleted human factor VIII cDNA was inserted into a cassette containing either spacer sequence (pDLZ2) or Enhancer I (EnhI) of hepatitis B virus and spacer sequence (pDLZ6) (Guo et al. (1991) J. Virology 65:6686). The sequence of pDLZ6 is presented in FIG. 1 (SEQ ID NO:1) along with the amino acid sequence of the B-domain deleted human factor VIII protein (also shown in SEQ ID NO:2). The first 19 amino acid residues represent a signal peptide, which is cleaved off before the B-domain deleted human factor VIII precursor is translocated into the endoplasmic reticulum. The B-domain deleted human factor VIII cDNA in pDLZ6 was replaced with EGFP cDNA from pTR-EGFP (R. Haberman, UNC Gene Therapy Center, Chapel Hill, N.C.) to construct pDLZ8. All constructs employ the Tk polyadenylation signal, and flanked using the AAV ITRs from pAAV/cFIX.

The pDLZ6 construct comprises two ITRs, at about nucleotide (nt) positions 1-146 and 4916-5084 of FIG. 1 (and SEQ ID NO:1), a hepatitis B virus EnhI enhancer element at about nucleotide positions 150-278, spacer sequence at about nucleotide positions 279-399, B-domain deleted human factor VIII cDNA at about nucleotide positions 419-4835, and a Tk polyA sequence at about nucleotide positions 4804-4914.

### EXAMPLE 2

Cells and Culture

293, HeLa, and HepG2 cells were cultured in Dulbecco's modified eagles media (DMEM, Gibco/BRL, Gaithersburg, Md.) with 10% fetal bovine serum (FBS, Gibco/BRL, Gaithersburg, Md.), with or without antibiotics (penicillin and streptomycin), at 37° C. and 5% CO2. FBS was heat-inactivated at 55° C. for 30 minutes. Under these conditions, factor VIII protein and activity could not be detected in FBS.

### EXAMPLE 3

rAAV Production and Purification

rAAV was generated using a three plasmid transfection scheme. Briefly, subconfluent 293 cells were co-transfected with the rAAV vector plasmid, AAV helper plasmid pXX2 (Xiao et al. (1998) J. Virology 72:2224), and adenovirus helper plasmid pXX6 using calcium phosphate precipitation. Forty-eight hours post-transfection, the cells were harvested, lysed by 3-cycles of freeze-thawing, and sonicated to release the rAAV virion particles. Following ammonium-sulfate precipitation, the virus particles were purified and concentrated by cesium density gradient centrifugation twice. Viral particles were titered by dot-blot; the rAAV/human factor VIII peak gradient fractions were pooled, dialyzed against phosphate buffer saline (PBS), and stored at −20° C.

### EXAMPLE 4

In vitro Expression of B-domain Deleted Human Factor VIII

2×10⁵ of 293 or HepG2 cells were plated in each well of 6-well plates. Twenty-four hours post-plating, cells were transduced with rAAV virus particles/cell (MOI=10), with or without adenovirus (MOI=1) for 1 hour. The cell media were harvested for analysis and replaced with fresh media every 24 hours post-infection. All the media/serum used for assaying human factor VIII expression and function were screened free of factor VIII.

### EXAMPLE 5

Protein Function and Inhibitor Assay for Human Factor VIII

rAAV-originated human factor VIII protein was detected by Enzyme-Linked Immunosorbent Assay (ELISA. Briefly, monoclonal sheep anti-human factor VIII antibody (Affinity Biological, Inc., Canada) was used as capture antibody. Peroxidase-conjugated sheep anti-human factor VIII antibody (Affinity Biological, Inc., Canada) was used as secondary antibody. The factor VIII levels were calculated according to the standard curve derived from serial dilution of the pooled normal human plasma (UCRP, Fisher Scientific). The reproducible sensitivity of the ELISA for human factor VIII was determined to be 0.3 ng/ml.

Function of the rAAV-originated B-domain deleted factor VIII was tested by the activated partial thromboplastin time (APTT) and Coatest (Chromgenix AB, Sweden). APTT was performed, except using factor VIII-deficient plasma rather than FIX-deficient plasma (Pacific Hemostasis). Coatest was performed following manufacturer's instructions. A serial dilution of pooled normal human plasma was used to generate the standard curve of factor VIII activity.

The Bethesda inhibitor assay (BIA) was used to detect anti-human factor VIII inhibitors in mouse serum (Kasper et al. (1975) Thrombosis et Diathesis Haemorrhagica 34:612). Briefly, mouse plasma was incubated at 55° C. for 30 minutes to inactivate endogenous murine factor VIII. The serial dilutions of the treated mouse plasma were then mixed with an equal volume of pooled normal human plasma (UCRP, Fisher Scientific) and incubated at 37° C. for 2 hours. APTT was performed to determine the residual factor VIII activity in the UCRP incubated with the inactivated mouse plasma. The anti-human factor VIII inhibitor titer was calculated from the residual factor VIII activity of each sample according to the established BIA standard curve.

### EXAMPLE 6

Animal Care and Manipulation Procedure

The mice were maintained-at the animal facilities at the University of North Carolina at Chapel Hill in accordance with the guidelines of the UNC Institutional Animal Care and Use Committee. Each animal was weighed and sedated using a mixture of ketamine (100 mg/kg) and xylanine (5 mg/kg) prior to virus administration. Under a dissecting microscope, a 1-cm vertical midline abdomen incision was made. 2×10¹⁰ or 2×10 ¹¹ particles of rAAV/DLZ6 or rAAV/DLZ8 in 200-400 μl of phosphate buffered saline (PBS) was injected to liver via portal vein using Harvard Apparatus pump 22 in 2-5 minutes. Blood was collected via the retro-orbital plexus and the plasma stored at −80° C. Tissues/organs were collected for histology and DNA/RNA analyses of three mice sacrificed at week 30 post-injection. Tissues collected included liver, spleen, kidney, testis, heart, brain, spinal cord, intestine, muscle, lymph nodes, and bone marrow. Tissues were either frozen at −80° C. (for DNA and RNA isolation) or fixed in 10% neutral-buffered formalin overnight before processing.

### EXAMPLE 7

DNA Isolation and Analysis

High molecular weight genomic and low molecular weight DNA (Hirt) were isolated and used for Southern Blot and DNA PCR. 29.5 pg, 5.9 pg, 1.18 pg, 0.118 pg, and 0.059 pg of plasmid pDLZ6 were added to 20 μg genomic DNA from control mouse liver produced copy number standard, respectively equivalent to 5, 1, 0.2, 0.02 and 0.01 copies of rAAV/DLZ6 vector genome per murine liver cell. The genomic DNA was digested with restriction enzyme SphI, which cuts the plasmid pDLZ6 internal to each ITR, releasing a 4.6 kb DLZ6 genome, and then separated by agarose gel. The blot was hybridized with ³²P-labeled human factor VIII probes.

A Sense primer (5′-AACCTTTACCCCGTTGCTCG-3′) and antisense primer (5′-GTCTTTTTGTACACGACTGAGG-3′) were used to amplify a 450 bp rAAV/DLZ6 vector unique fragment. The PCR conditions were 95° C. for 5 minutes followed by 30 cycles with 95° C. for 2 minutes, 50° C. for 1 minute, 72° C. for 1 minute.

### EXAMPLE 8

RNA Extraction, Northern Blot and Reverse Transcription (RT) PCR

Total cellular RNA extracted from cultured cells or frozen mouse tissues was used for Northern Blot or RT-PCR in a similar. A sense primer (5′-TTCTCCCCAATCCAGCTGG-3′) and antisense primer (5′-GAGTTATTTCCCGTTGATGG-3′) were used to amplify a 534 bp unique human factor VIII cDNA fragment. The PCR conditions were 95° C. for 2 minutes, followed with 30 cycles using: 95° C. for 1 minute, 55° C. for 1 minute, 72° C. for 1 minute. A pair of β-actin primers was used as an internal control of RT/PCR for each sample described.

### EXAMPLE 9

Histological Analysis

Formalin-fixed tissues were alcohol dehydrated and paraffin embedded. Tissues were sectioned at 6 μm each, deparaffinized in xylene, rehydrated through graded ethanol, and either stained with hematoxylin and eosin (H & E).

### EXAMPLE 10

Packaging of rAAV B-domain Deleted Human Factor VIII

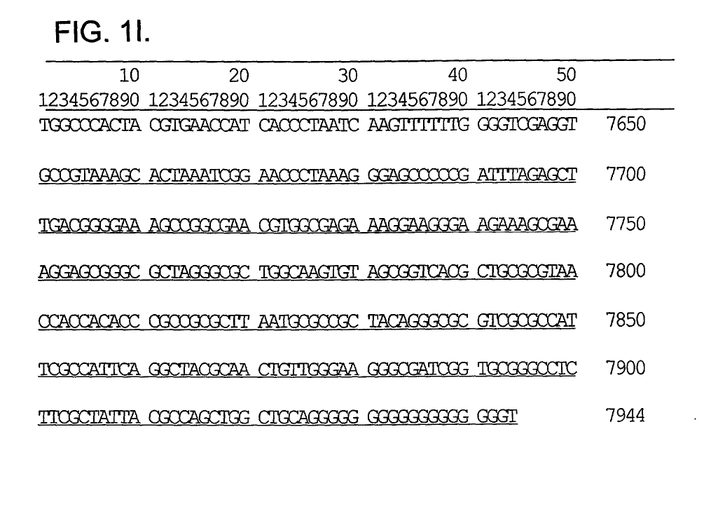

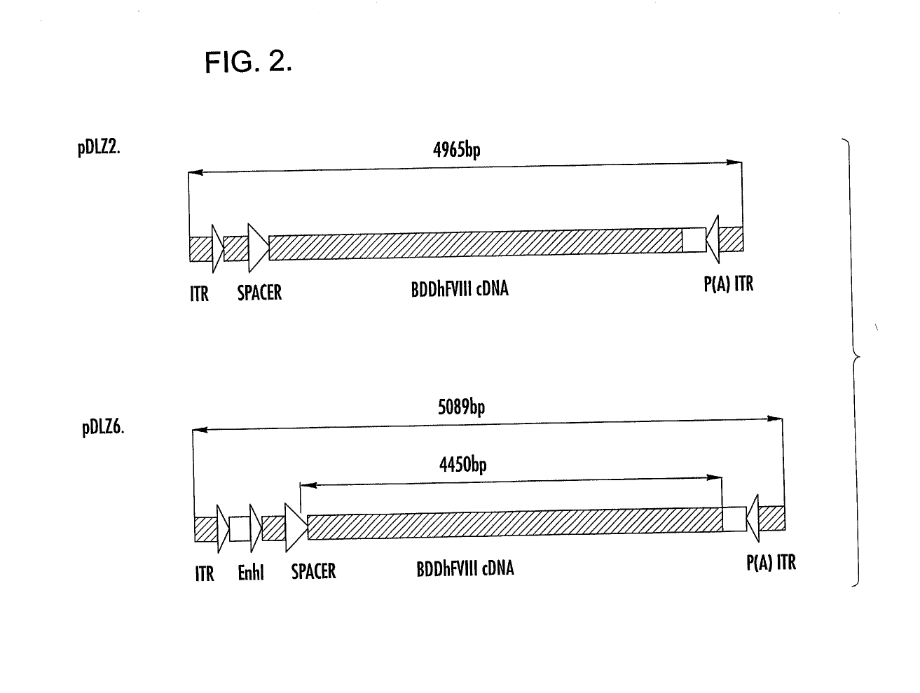

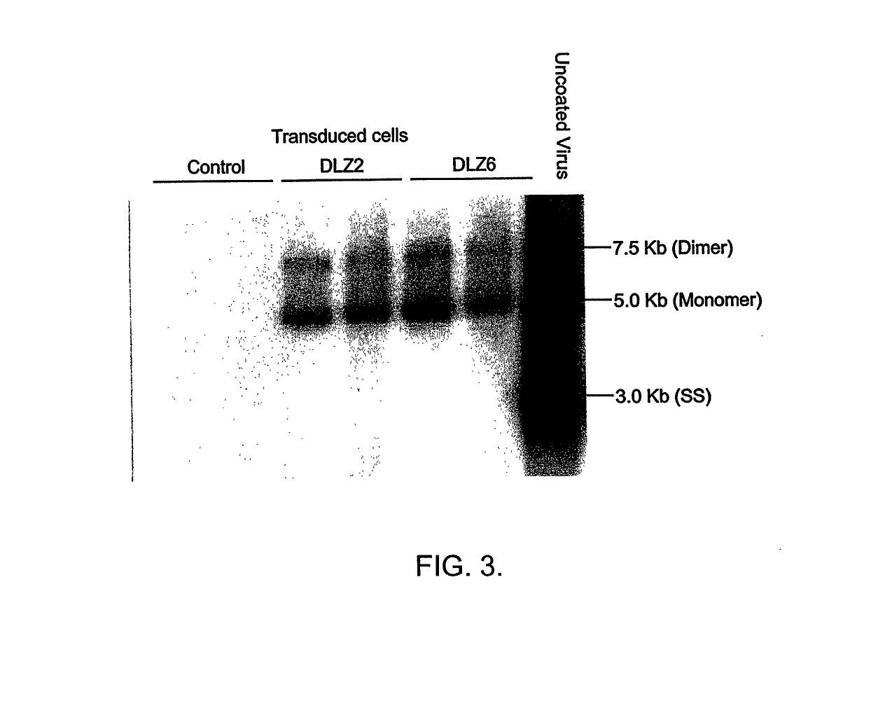

Two rAAV vectors expressing B-domain deleted human factor VIII, pDLZ2 and pDLZ6 (FIG. 2), were constructed to test the utility of the Hepatitis B virus EnhI enhancer element. Over 10¹² rAAV/DLZ6 or rAAV/DLZ2 particles per milliliter were produced using triple plasmid transfection and cesium chloride density gradient centrifugation. To confirm the replication of rAAV virions, low molecular weight viral DNA was isolated following transduction of HeLa or HepG2 cells with rAAV (MOI=10) and adenovirus type 5 (MOI=1). As shown in FIG. 3, the expected monomer and dimer replication forms of rAAV/DLZ6 and rAAV/DLZ2 were detected using a probe specific for the transgene. Isolation of rAAV/DLZ6 virion DNA confirmed that the expected monomer size was packaged (FIG. 3). Following transduction, rAAV/DLZ6 containing the EnhI sequence produced a 30-fold increase in mRNA transcript in HeLa and HepG2 as compared to rAAV lacking the enhancer element (data not shown).

Based on these results, we performed fucker VIII functional assays using vector derived from pDLZ6. human factor VIII protein expression was performed by ELISA measurement of factor VIII protein from cell media harvested at 24 hours following transfection and transduction. Assessment of functional human factor VIII was performed using APTT and Coatest assays (see Table 1). Thus despite its greater than wild-type size, recombinant virus was efficiently packaged and produced functional B-domain deleted human factor VIII. Based on these results, rAAV/DLZ6 was used for in vivo analysis.

TABLE 1
In vitro Expression of B-domain deleted
human factor VIII from AAV Vectors

| | Antigen Assay | Functional | Assay |
|---|---|---|---|
| | ELISA | APTT | Coatest |
| Transfection | 5.6 ng/ml | 25% | 28 mu/ml |
| Transduction | 15 ng/ml | 40% | 72 mu/ml |

** 1 × 10⁶ 293 cells were transduced with rAAV/DLZ6 or rAAV/DLZ8 (EGFP) at MOI = 10. Media were harvested at 24 hours for human factor VIII assay. The media overlay 293/EGFP was used as control. UCRP served as the standard, which is equivalent to 200 ng/ml human factor VIII antigen and 1000 mu/ml Coatest activity. APTT refers to the percent of normal factor VIII activity. Results are expressed as the mean of three experiments, each performed in triplicate.

### EXAMPLE 11

Long-term Expression of Human Factor VIII in Mice

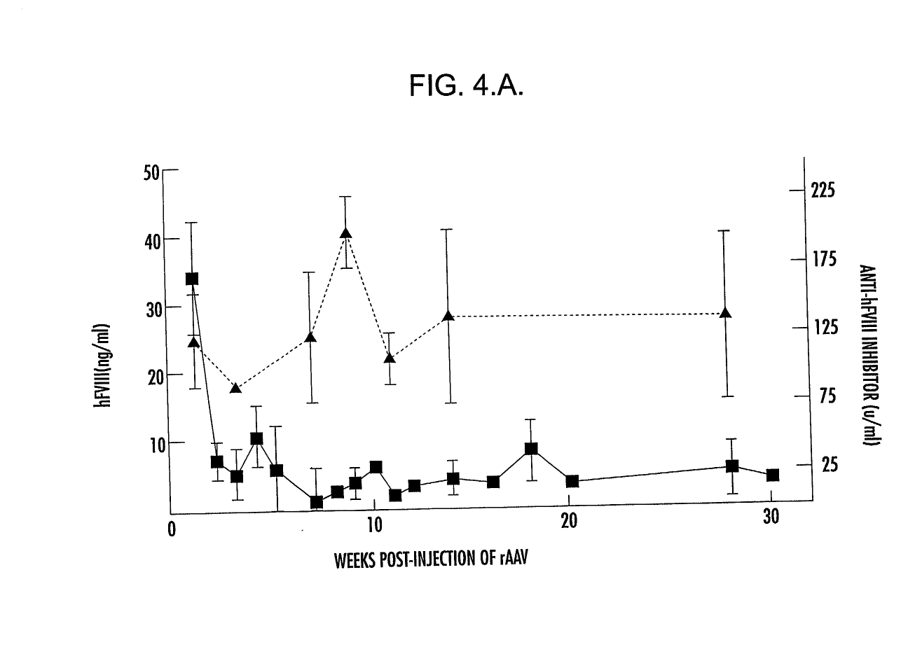

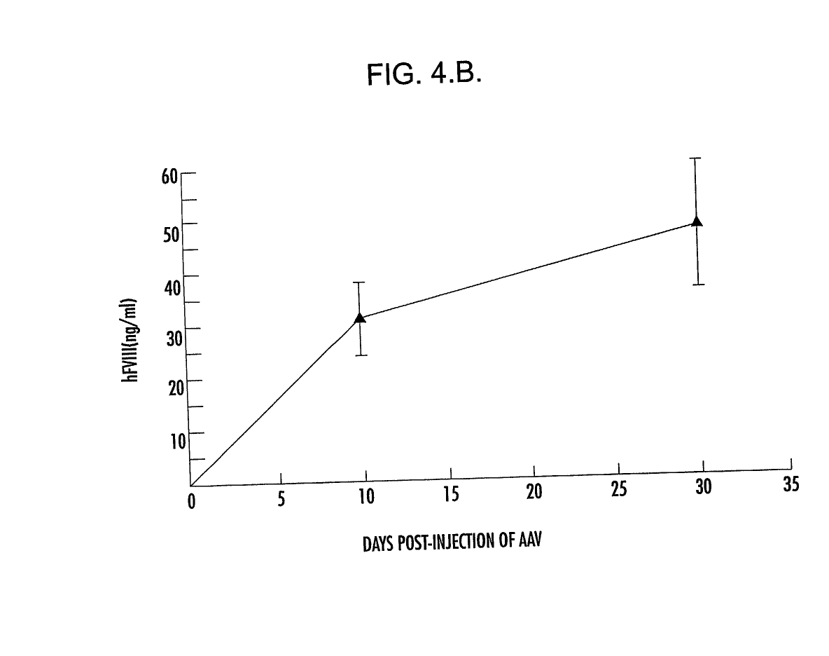

rAAV/DLZ6 was injected into the portal vein of 4-week-old male mice or 6-week-old NOD/scid mice. Blood samples were collected via the retro-orbit plexus biweekly. B-domain deleted human factor VIII protein was not detected in the plasma of 2 mice receiving 2×10¹⁰ rAAV/DLZ6 until 4 weeks post-injection of the AAV (data not shown). Once detected, the human factor VIII levels remained at 2-3% of normal human levels factor VIII level (200 ng/ml) for over 11 months. In contrast, a mean of 42 ng/ml of B-domain deleted human factor VIII or 21% of normal human factor VIII level was detected in the plasma of 4 mice receiving 2×10¹¹ rAAV/DLZ6 at 1 week post-injection (FIG. 4, Panel A). High titer anti-human factor VIII inhibitor was detected in the plasma of all of the mice receiving rAAV/DLZ6 as early as 1 week post-injection (see FIG. 4, Panel A). The anti-human factor VIII inhibitor titer increased to a maximum titer at 9 to 12 weeks post-injection (FIG. 4, Panel A). The appearance of inhibitor coincided with the decrease in B-domain deleted human factor VIII plasma protein. As expected, neither B-domain deleted human factor VIII nor anti-human factor VIII inhibitor were detected in the plasma of control mice receiving rAAV expressing the EGFP transgene (data not shown).

In order to adequately assess the expression of B-domain deleted human factor VIII protein, immuno-incompetent NOD/scid mice received 1.5×10¹¹ virus via portal vein injection. Plasma levels of B-domain deleted human factor VIII determined by ELISA reached 35 ng/ml (17% of normal level) on day 10 post-injection and increased to 55 ng/ml (27% of normal level) (FIG. 4, Panel B). As expected, B-domain deleted human factor VIII was not detected in the plasma of mock infected scid mice (data not shown).

### EXAMPLE 12

rAAV Vector Spread and Histologic Analysis

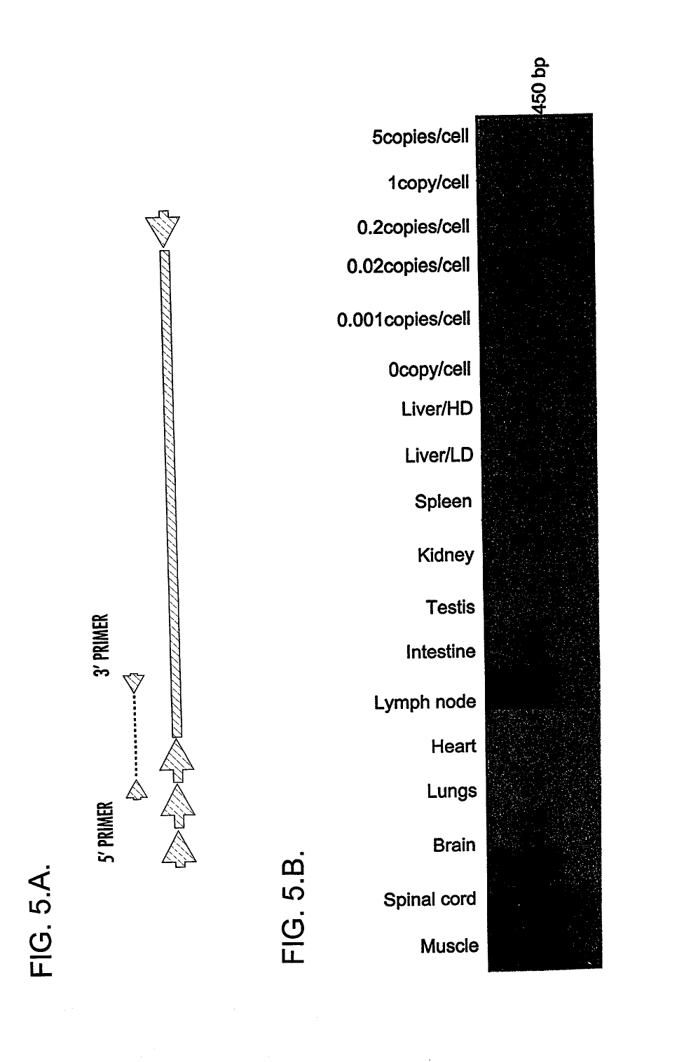

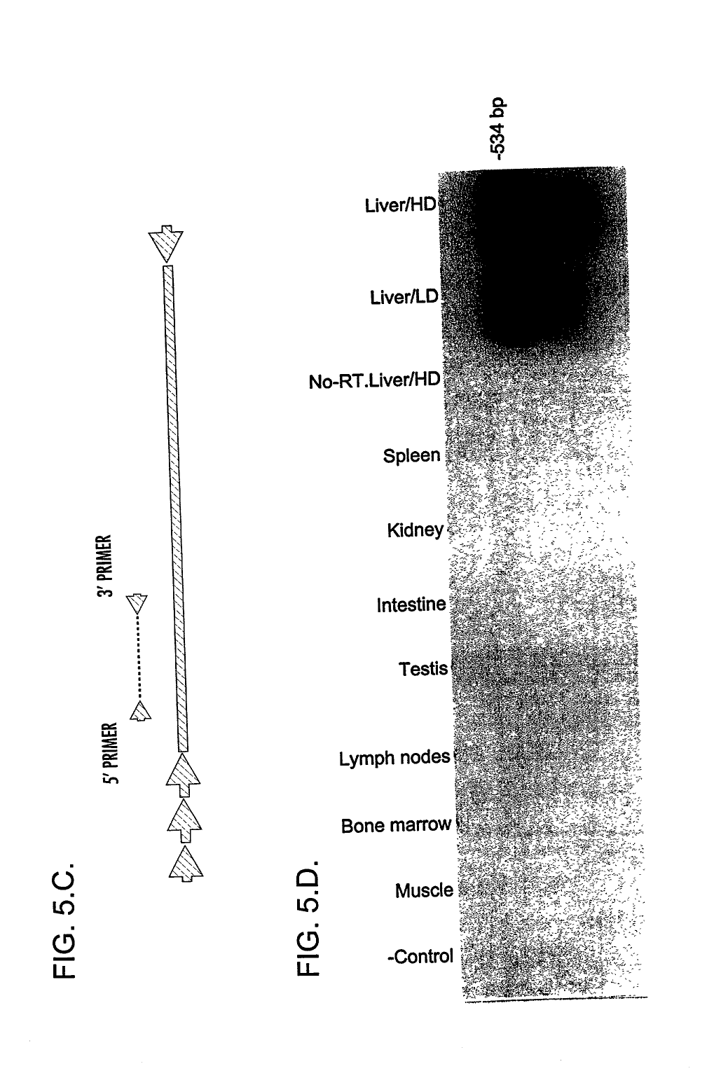

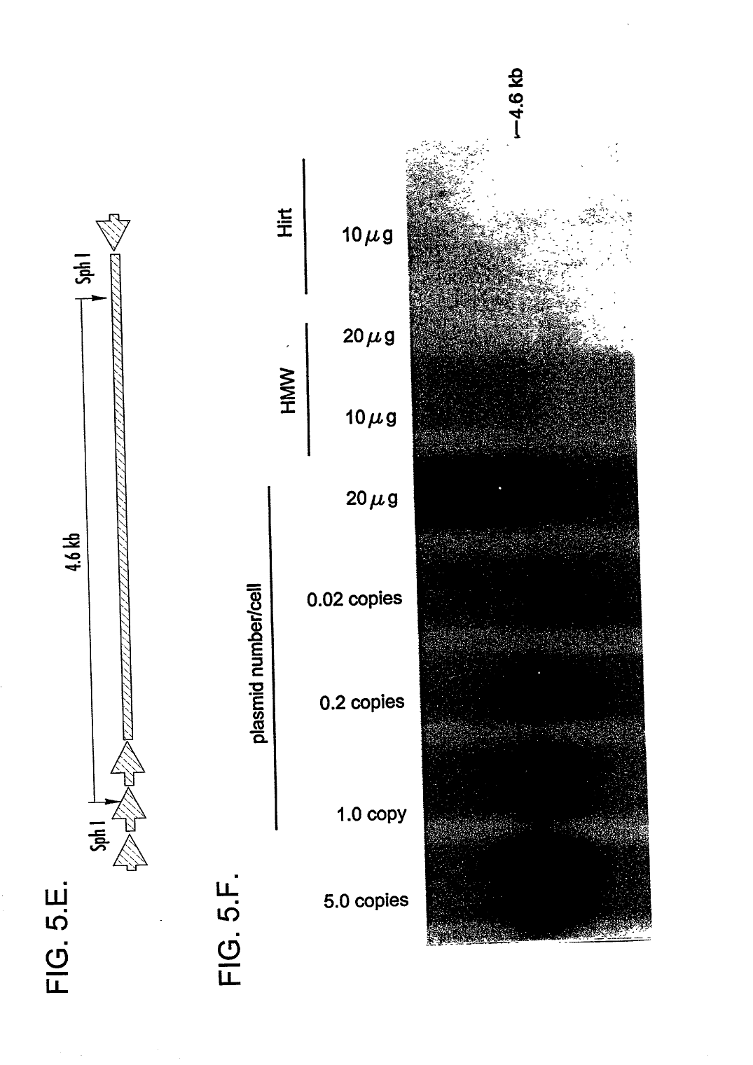

The mice receiving rAAV vector were sacrificed at 30 weeks post-injection. Peripheral blood, liver, spleen, lymph nodes, kidney, intestine, testis, skin, muscle, heart, lungs, aorta, bone marrow, brain and spinal cord were analyzed to determine vector spread following systemic administration. DNA PCR utilizing primer pairs specific for the vector DLZ6 amplified a 450-bp product. Vector genome was detected only from liver samples 30 weeks after portal vein injection (FIG. 5, Panel A). RT-PCR employed a pair of primers which amplify a 534 bp fragment of B-domain deleted human factor VIII cDNA. Only RNA isolated from the liver generated the appropriate PCR product, confirming the DNA PCR result (FIG. 5, Panel B). Amplification of a 250 bp β-actin fragment was utilized as internal control for RT/PCR showed intact and equal amount of RNA were used for each sample in RT-PCR (data not shown). By using both DNA PCR and Southern blot analysis, an estimated 0.05 copies of rAAV/DLZ6 genome per cell were present at 30 weeks post-transduction in animals given 2×10¹¹ rAAV particles (FIG. 5, Panels A & C). This result is in agreement with previous reports ((Snyder et al. (1999) Nature Medicine 5:64; Xiao et al (1998) J. Virology 72:10222). No significant pathology was observed in the licker, spleen, GI tract, gonads, brain, heart, and lungs (data not shown).

### EXAMPLE 13

rAAV Molecular Analysis in Liver Cells

At the time of sacrifice, 30 weeks, low molecular weight DNA (Hirt DNA) and high molecular weight genomic DNA were isolated from several organs of the mice receiving rAAV/DLZ6. Using the restriction enzyme Sph I, which cuts internal to each ITR, and Southern blotting unrearranged rAAV/DLZ6 genome were detected only in the high molecular weight fraction (FIG. 5, Panel C). Approximately 0.05 vector genome copies/cell were detected in the high molecular weight DNA fraction. DNA PCR confirmed that the rAAV/DLZ6 vector genome signal could not be detected in the Hirt DNA fraction (data not shown). The sensitivity of the PCR assay is 0.001 copies/cell.

### EXAMPLE 14

Phenotypic Correction in Factor VIII Knock-Out Mice

rAAV/DLZ6 is administered to mice in which the gene encoding factor VIII has been “knocked out” by homologous recombination, thereby producing a phenotype corresponding to hemophilia A. Mice are administered either 2×10¹⁰ or 2×10¹¹ particles of rAAV/DLZ6 or a control vector via portal vein injection as described in the previous Examples.

Hepatic expression of B-domain deleted human factor VIII is determined as described in the previous Examples. In addition, plasma levels of B-domain deleted human factor VIII and factor VIII inhibitors are monitored over time, also as described above. Functional assays of factor VIII activity (e.g., Coatest) are also carried out to determine functional B-domain deleted human factor VIII protein expression in plasma. The rAAV/DLZ6-treated mice are monitored over time for phenotypic changes due to expression of the B-domain deleted human factor VIII, i.e., amelioration or correction of phenotypic traits associated with hemophilia (for example, improved clotting time).

In this manner, long-term hepatic expression of B-domain deleted human factor VIII using a rAAV vector (Example 11) is correlated with phenotypic improvement in hemophiliac animals.

### EXAMPLE 15

Phenotypic Correction in Hemophiliac Dogs

Hemophiliac dogs are administered a rAAV vector carrying a B-domain deleted canine factor VIII (canine factor VIII). The B-domain deleted canine factor VIII expression cassette is essentially as described in Example 1 for the human factor VIII expression cassette and includes flanking AAV ITRs, EnhI enhancer, noncoding sequence, and Tk poly(A) sequence. Plasmid pDLZ10 encodes the canine factor VIII expression cassette. The nucleotide sequence of pDLZ10 is shown in FIG. 7 along with the amino acid sequence of the B-domain deleted canine factor VIII encoded thereby. This construct comprises two ITRs, at about nucleotide (nt) positions 1-144 and 4885-5048 of FIG. 1 (SEQ ID NO:1), a hepatitus B virus EnhI enhancer element at about nt positions 149-278, spacer sequence at about nt positions 279-399, BBD canine factor VIII cDNA at about nt positions 428-4790, and a polyA sequence at about nt positions 4804-4884. Dogs are infused with 10¹³ or 10¹⁴ particles of rAAV/canine factor VIII or a control vector by portal vein. In the same or a separate study, the same titer of rAAV vector is administered by direct hepatic vessel injection.

Hepatic expression of B-domain deleted canine factor VIII is determined as described in the previous Examples. In addition, plasma levels of B-domain deleted canine factor VIII and factor VIII inhibitors are monitored over time, also as described above. Functional assays of factor VIII activity (e.g., Coatest) are also carried out to determine functional B-domain deleted canine factor VIII protein expression in plasma. The rAAV/B-domain deleted canine factor VIII treated dog style are monitored over time for phenotypic changes due to expression of the B-domain deleted canine factor VIII, i.e., amelioration or correction of phenotypic traits associated with hemophilia (for example, improved clotting time).

In this manner, delivery of B-domain deleted canine factor VIII to the liver of hemophiliac dogs using a rAAV vector is evaluated for the treatment of hemophilia A.

### EXAMPLE 16

Generation of a Stable Producer Cell Line for rAAV/B-domain Deleted Factor VIII

Generally, rAAV producer cell lines are generated by transfection of cells with vector plasmid, followed by selection with antibiotics (typically G418, hygromycin, or histidinol) and cloning of individual colonies. Colonies are first screened for vector replication. Clones showing high level replication of vector following adenovirus infection are then tested for production of infectious vector.

Plasmid B-domain deletedfactor VIII (30 μg) was transfected into the Hela C12 packaging cell line by electroporation (Potter et al., 1984, Proc. Natl. Acad. Sci. USA 79:7161-7165). The C12 cell line contains the AAV2 rep and cap genes that are transcriptionally quiescent until induction upon infection with adenovirus helper (Clark et al., 1995; Clark et al., 1996, Gene Therapy 3:1124-1132). Twenty four hours post-transfection, the cells were trypsinized and replated in 100 mm plates at densities ranging from 5×103 to 5×104 cells per plate. The cells were subjected to selection in DMEM containing 10% fetal bovine serum and 300 μg/ml hygromycin B. Drug-resistant cell clones were isolated, expanded and their ability to produce infectious AAV factor VIII vectors was tested and compared in an infectivity assay as described in Atkinson et al., 1998, Nucleic Acid Res. 26:2821-2823. One such producer cell clone (C12-55) was further used for production of vector. Production, purification and titration were carried out essentially as described herein and as generally described in Atkinson et al. (WO 99/11764).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed is:

1. A recombinant adeno-associated virus (rAAV) vector comprising a heterologous nucleotide sequence encoding B-domain deleted factor VIII operably linked with at least one enhancer and at least one promoter.

2. The rAAV vector of claim 1, wherein said rAAV vector further comprises spacer DNA.

3. The rAAV vector of claim 1, wherein said rAAV is selected from the group consisting of AAV serotype 1, serotype 2, serotype 3, serotype 4, and serotype 5.

4. The rAAV vector of claim 1, wherein said B-domain deleted factor VIII is a human B-domain deleted factor VIII.

5. The rAAV vector of claim 4, wherein said heterologous nucleotide sequence encodes a B-domain deleted factor VIII having the amino acid sequence set forth in SEQ ID NO:2.

6. The rAAV vector of claim 4, wherein said heterologous nucleotide sequence comprises the sequence given as about nucleotides 419 to 4835 of the nucleotide sequence set forth in SEQ ID NO:1.

7. The rAAV vector of claim 1, wherein said promoter is an AAV ITR.

8. A pharmaceutical formulation comprising the rAAV vector of claim 1 in a pharmaceutically acceptable carrier.

9. A recombinant adeno-associated virus (rAAV) vector comprising a heterologous nucleotide sequence encoding factor VIII operably linked with a liver-preferred expression control element.

10. The rAAV vector of claim 9, wherein said heterologous nucleotide sequence comprises the sequence given as about nucleotides 419 to 4835 of the nucleotide sequence set forth in SEQ ID NO:1.

11. The rAAV vector of claim 9, wherein said liver-preferred expression control element comprises at least one enhancer selected from the group consisting of the α1 microglobulin/bikunin enhancer, the hepatitis B virus EnhI enhancer, the hepatitis B virus EnhII enhancer, the human albumin E_{1.7} enhancer, and the human albumin E₆ enhancer.

12. The rAAV vector of claim 9, wherein said liver-preferred expression control element comprises the hepatitis B virus Enhli enhancer given as about nucleotides 419 to 4835 of the nucleotide sequence set forth in SEQ ID NO:1.

13. The rAAV vector of claim 9, wherein said liver-preferred expression control element comprises at least one promoter selected from the group consisting of the hepatitis B virus core promoter, the mouse albumin promoter, the human U1 snRNA promoter, and the herpes simplex virus thymidine kinase promoter.

14. The rAAV vector of claim 9, wherein said liver-preferred expression control element comprises at least one transcription factor binding site selected from the group consisting of a TATA box, a CAAT box, a GC box, an ATF box, a C/EBP binding site, an HNF1 binding site, an HNF2 binding site, an HNF3 binding site, an HNF4 binding site, and a TGT3 binding site.

15. The rAAV vector of claim 9, wherein said heterologous nucleotide sequence further comprises sequences encoding a promoter and a polyadenylation sequence.

16. The rAAV vector of claim 9, wherein said heterologous nucleotide sequence comprises the sequence given as about nucleotides 150 to 4914 of the nucleotide sequence set forth in SEQ ID NO:1.

17. The rAAV vector of claim 9, wherein said heterologous nucleotide sequence encodes the amino acid sequence set forth in SEQ ID NO:2.

18. A recombinant adeno-associated virus (rAAV) vector comprising a heterologous nucleotide sequence encoding a B-domain deleted factor VIII operably linked with an enhancer, wherein said nucleotide sequence is selected from the group consisting of:
(a) the nucleotide sequence given as nucleotides 419 to 4835 of the nucleotide sequence set forth in SEQ ID NO:1,
(b) a nucleotide sequence that hybridizes to the nucleotide sequence of (a) under conditions of high stringency and which encodes a B-domain deleted factor VIII, and
(c) a nucleotide sequence that that differs from the nucleotide sequences of (a) and (b) above due to the degeneracy of the genetic code, and which encodes a B-domain deleted factor VIII.

19. The rAAV vector of claim 18, wherein said rAAV further comprises spacer DNA.

20. A composition comprising a population of at least about 10¹² recombinant adeno-associated virus (rAAV) vector particles comprising a heterologous nucleotide sequence encoding B-domain deleted factor VIII.

21. A method of delivering a nucleotide sequence encoding B domain-deleted factor VIII to a cell comprising contacting the cell with a recombinant adeno-associated virus (rAAV) vector comprising a heterologous nucleotide sequence encoding B-domain deleted factor VIII operably linked with a liver-preferred expression control element.

22. The method of claim 21, wherein the contacting is carried out in vitro.

23. The method of claim 21, wherein the contacting is carried out in vivo.

24. The method of claim 21, wherein the cell is selected from the group consisting of neural cells, liver cells, muscle cells, retinal cells, epithelial cells, fibroblast cells, germ cells, bone marrow cells, hematopoietic stem cells, spleen cells, pancreas cells, and cells of the central nervous system.

25. The method of claim 24 wherein the cell is a liver cell.

26. The method of claim 21, wherein the cell is a human cell.

27. The method of claim 21, wherein said liver-preferred expression control element comprises at least one enhancer selected from the group consisting of the α1 microglobulin/bikunin enhancer, the hepatitis B virus EnhI enhancer, the hepatitis B virus EnhII enhancer, the human albumin E_{1.7} enhancer, and the human albumin E₆ enhancer.

28. The method of claim 21, wherein said liver-preferred expression control element comprises the hepatitis B virus EnhI enhancer given as about nucleotides 419 to 4835 of the nucleotide sequence set forth in SEQ ID NO:1.

29. The method of claim 21, wherein said liver-preferred expression control element comprises at least one promoter selected from the group consisting of the hepatitis B virus core promoter, the mouse albumin promoter, the human U1 snRNA promoter, the herpes simplex virus thymidine kinase promoter.

30. The method of claim 21, wherein said liver-preferred expression control element comprises at least one transcription factor binding site selected from the group consisting of a TATA box, a CAAT box, a GC box, an ATF box, a C/EBP binding site, an HNF1 binding site, an HNF2 binding site, an HNF3 binding site, an HNF4 binding site, and a TGT3 binding site.

31. The method of claim 21, wherein said tAAV vector additionally comprises at least one AAV ITR operably linked to said nucleotide sequence encoding B-domain deleted factor VIII such that said AAV ITR drives expression of said nucleotide sequence encoding B-domain deleted factor VIII.

32. The method of claim 21, wherein the B-domain deleted factor VIII is a human B-domain factor VIII.

33. The method of claim 32, wherein said heterologous nucleotide sequence encodes a B-domain deleted factor VIII having the amino acid sequence set forth in SEQ ID NO:2.

34. The method of claim 33, wherein said heterologous nucleotide sequence comprises the sequence given as about nucleotides 419 to 4835 of the nucleotide sequence set forth in SEQ ID NO:1.

35. A method of delivering a nucleotide sequence encoding a B-domain deleted factor VIII to a cell comprising contacting the cell with a recombinant adeno-associated virus (rAAV) vector comprising a heterologous nucleotide sequence encoding a B-domain deleted factor VIII selected from the group consisting of:
(a) the nucleotide sequence given as nucleotides 419 to 4835 of the nucleotide sequence set forth in SEQ ID NO:1,
(b) a nucleotide sequence that hybridizes to the nucleotide sequence of (a) under conditions of high stringency and which encodes a B-domain deleted factor VIII, and
(c) a nucleotide sequence that that differs from the nucleotide sequences of (a) and (b) above due to the degeneracy of the genetic code, and which encodes a B-domain deleted factor VIII.

36. A method of delivering a nucleotide sequence encoding B-domain deleted factor VIII to a cell comprising contacting the cell with a composition comprising a population of recombinant adeno-associated virus (AAV) vectors comprising a heterologous nucleotide sequence encoding B-domain-deleted factor VIII, and further wherein said composition has a titer of at least about 10⁸ infectious units per milliliter.

37. A method of enhancing blood coagulation in a subject in need thereof comprising administering a recombinant adeno-associated virus (rAAV) vector comprising a heterologous nucleotide sequence encoding B-domain deleted factor VIII to the subject in an amount sufficient to enhance blood coagulation.

38. The method of claim 37, wherein at least about 2×10¹⁰ particles of the rAAV vector are administered to the subject.

39. The method of claim 37, wherein the subject is a mammalian subject.

40. The method of claim 39, wherein the subject is a human subject.

41. The method of claim 40, wherein the rAAV vector is administered by a route selected from the group consisting of oral, rectal, transmucosal, transdermal, inhalation, intravenous, subcutaneous, intradermal, intracranial, intramuscular, and intraarticular administration.

42. The method of claim 41, wherein the rAAV is administered to the liver of the subject.

43. The method of claim 44, wherein the rAAV is administered to the liver by a route selected from the group consisting of intravenous administration, intraportal administration, intrabiliary administration, intra-arterial administration, and direct injection into the liver parenchyma.

44. The method of claim 37, wherein the rAAV further comprises a liver-preferred expression control element operably linked with the heterologous nucleotide sequence encoding factor VIII.

45. The method of claim 44, wherein said liver-preferred expression control element comprises at least one enhancer selected from the group consisting of the α1 micorglobulin/bikunin enhancer, the hepatitis B virus E_{1.7} enhancer, the hepatitis B virus EnhII enhancer, the human albumin E_{1.7} enhancer, and the human albumin E₆ enhancer.

46. The method of claim 45, wherein the liver-preferred expression control element is a hepatitis B virus enhancer element EnhI or a hepatitis B virus enhancer element EnhII.

47. The method of claim 37, wherein the B-domain deleted factor VIII is a human B-domain deleted factor VIII.

48. The method of claim 47, wherein the heterologous nucleotide sequence encodes a B-domain deleted factor VIII having the sequence given in SEQ ID NO:2.

49. The method of claim 48, wherein the heterologous nucleotide sequence encodes the amino acid sequence set forth in SEQ ID NO:2.

50. A method of treating hemophilia A comprising administering to a hemophiliac subject a biologically effective amount of a recombinant adeno-associated virus (rAAV) vector comprising a heterologous nucleotide sequence encoding B-domain deleted factor VIII, wherein said B-domain deleted factor VIII is expressed at therapeutically effective amounts.

51. A method of treating hemophilia comprising administering to the liver of a hemophiliac subject, a biologically effective amount of a recombinant adeno-associated virus (rAAV) vector comprising a heterologous nucleotide sequence encoding B-domain deleted factor VIII.

52. The method of claim 51, wherein the liver expresses the encoded B-domain deleted factor VIII, which is secreted into the blood in a therapeutically effective amount.

53. A method of administering B-domain deleted factor VIII to a subject comprising administering a cell expressing B-domain deleted factor VIII to the subject, wherein the cell has been produced by a method comprising contacting the cell with a recombinant adeno-associated virus (rAAV) vector comprising a nucleotide sequence encoding B-domain deleted factor VIII.

54. The method of claim 53, wherein the cell is selected from the group consisting of hematopoietic stem cells, liver cells, fibroblasts, epithelial cells, spleen cells, pancreatic cells, keratinocytes, endothelial cells, myoblasts, and neural cells.

55. A method of producing a high-titer stock of a recombinant adeno-associated virus (rAAV) vector comprising
(a) infecting a packaging cell with a rAAV vector comprising a heterologous nucleotide sequence encoding factor VIII,
(b) allowing the rAAV genome to replicate and be encapsidated by the packaging cell, and
(c) collecting the rAAV particles to form a rAAV stock;
wherein the titer of the rAAV stock is at least about 10⁶ infectious units per milliliter.

56. The method of claim 55, wherein the heterologous nucleotide sequence encoding factor VIII is operably linked with a liver-preferred expression control element.

57. A virus stock produced by the method of claim 55.

58. A nucleotide sequence encoding B-domain deleted factor VIII operably linked with a hepatitis virus expression control element.

59. The nucleotide sequence of claim 58, wherein said hepatitis virus expression control element is from a hepatitis B virus.

60. The nucleotide sequence of claim 59, wherein said hepatitis virus expression control element is a hepatitis B virus EnhI or EnhI enhancer.

61. The nucleotide sequence of claim 60, wherein said hepatitis virus expression control element is a hepatitis B virus EnhI enhancer.

62. The nucleotide sequence of claim 58, wherein said nucleotide sequence comprises the sequence given as about nucleotides 150 to 4835 of the nucleotide sequence set forth in SEQ ID NO:1.

63. The nucleotide sequence of claim 62, wherein said nucleotide sequence further comprises a promoter and a polyadenylation sequence.

64. The nucleotide sequence of claim 63, wherein said nucleotide sequence comprises the sequence given as nucleotides 150 to 4914 of the nucleotide sequence set forth in SEQ ID NO:1.

65. A vector comprising the nucleotide sequence of claim 58.

66. The vector of claim 65, wherein said vector is the plasmid disclosed herein as pDLZ6.

67. A cell containing the vector of claim 65.
